(19) United States
(12) Patent Application Publication
(10) Pub. No.: US 2026/0347478 A1
(43) Pub. Date: Nov. 5, 2026

(54) BISPECIFIC ANTIBODY AND USE THEREOF

(71) Applicant: HEFEI TG IMMUNOPHARMA CO., LTD., Hefei (CN)

(72) Inventors: Guoshuai Cao, Hefei (CN); Ying Cheng, Hefei (CN); Yangyang Li, Hefei (CN); Yuwei Wu, Hefei (CN)

(21) Appl. No.: 19/703,023

(22) Filed: Jun. 9, 2026

Related U.S. Application Data
(63) Continuation of application No. PCT/CN2024/136990, filed on Dec. 5, 2024.

(30) Foreign Application Priority Data
Dec. 11, 2023 (CN) 202311698119.6

(51) Int. Cl. C07K 16/28; A61K 39/00; A61P 35/00

(52) U.S. Cl. CPC C07K 16/2827; A61P 35/00; C07K 16/2809; A61K 2039/505; C07K 2317/31; C07K 2317/33; C07K 2317/522; C07K 2317/55; C07K 2317/565; C07K 2317/567; C07K 2317/622; C07K 2317/92

(57) ABSTRACT

Provided are a bispecific antibody targeting CD3 and PD-L1 and use thereof. The bispecific antibody includes a first antigen binding region; a second antigen binding region; and an Fc portion. The Fc portion includes a first Fc peptide fragment and a second Fc peptide fragment. The first antigen binding region includes a scFv fragment and a binding protein of a first molecule or fragment thereof. The binding protein of the first molecule or fragment thereof is linked to an N-terminus of the scFv fragment. A C-terminus of the scFv fragment is linked to an N-terminus of the first Fc peptide fragment. The second antigen binding region includes a binding protein of a second molecule or fragment thereof. The binding protein of the second molecule or fragment thereof is linked to an N-terminus of the second Fc peptide fragment. The first molecule and the second molecule are PD-L1.

### CROSS-REFERENCE TO RELATED APPLICATIONS

This application is a continuation of International Application No. PCT/CN2024/136990, filed on Dec. 5, 2024, which claims priority to Chinese Patent Application No. 202311698119.6, filed on Dec. 11, 2023. The disclosures of the aforementioned applications are hereby incorporated by reference in their entireties.

### STATEMENT REGARDING SEQUENCE LISTING

A Sequence Listing associated with this application is being filed concurrently herewith in XML format and is hereby incorporated by reference into the present specification. The XML file containing the Sequence listing is titled “Sequence_Listing.xml”, was created on Jun. 3, 2026, edited on Jun. 8, 2026, and is 28,606 bytes as measured in Microsoft Windows operating system.

### FIELD

The present disclosure belongs to the field of biomedicine technologies, and particularly, the present disclosure relates to a bispecific antibody and use thereof, more particularly, the present disclosure relates to an isolated nucleic acid, an expression vector, a recombinant cell, a pharmaceutical composition, and a kit and use thereof.

### BACKGROUND

Cancer is a class of diseases caused by unlimited proliferation and spread of abnormal cells. Cancer is a complex group of diseases, including many types of cancer, such as breast cancer, lung cancer, and colorectal cancer. Development of cancer usually involves multiple gene mutations and genetic abnormalities, leading to uncontrolled proliferation and invasive growth of cells. Traditional cancer treatment methods include surgical resection, radiotherapy, and chemotherapy. However, these treatment methods have many limitations, such as limited feasibility of surgical resection, toxic and side effects, and drug resistance. Therefore, researchers have been seeking new cancer treatment strategies, one of which is to use a bispecific antibody.

The bispecific antibody is an engineered antibody that is capable of binding two different antigens simultaneously. The bispecific antibody is usually composed of structural domains of two monoclonal antibodies and may simultaneously bind to an antigen on a surface of a tumor cell and an antigen on a surface of an immune cell (such as a T cell). The structure of such bispecific antibody enables them to link the immune cell and cancer cell, thereby promoting the attack and killing of the cancer cell by the immune cell. Use of the bispecific antibody has multiple of mechanisms and advantages in cancer treatment, including: activation of immune cell: the bispecific antibody can tightly bind the immune cell (such as the T cell and a natural killer cell) to the cancer cell, activating a killing function of the immune cell, and enhancing an attack force on the cancer cell; immune cell localization: the bispecific antibody can localize the immune cell to tumor tissue, increasing a local concentration of a drug, and reducing the toxic and side effects on a normal tissue; inhibition of tumor growth signaling pathway: some bispecific antibodies can inhibit a signaling pathway of tumor growth and metastasis by binding to the antigen on the surface of the tumor cell; and stimulation of an immune response: the bispecific antibody can activate the immune response, increase antigen presentation, T cell activation, and immunological memory, etc. the bispecific antibody, as an emerging therapeutic strategy, exhibits great potential in the field of cancer treatment. Therefore, there is an urgent need to develop a bispecific antibody with low toxicity and strong tumor-killing ability.

### SUMMARY

The present disclosure aims to solve at least one of the technical problems in the related art to some extent.

The present disclosure is completed based on the following findings.

CD3 and PD-L1-based bispecific antibody promotes T cell activation and kills tumors by recruiting T cells to local tumor site and bridging the T cells and the tumors. The bispecific antibody can guide the T cells to kill “cold tumors” without neoantigens. However, due to a strong binding ability of the bispecific antibody to the T cells, a severe cytokine storm and an excessive immune response may be triggered and thereby causing body damage and seriously possibly endangering life. It was unexpectedly found that the CD3 and PD-L1-based bispecific antibody with a structure of the present disclosure exhibits improved the killing activity on tumor cells compared with a bispecific antibody of a conventional format (CD3×PDL1 1:1), while reducing the binding ability of the bispecific antibody with the T cells. The above bispecific antibody can have advantages of better anti-tumor activity, higher safety and the like, and have high value in clinical application and drug development.

Therefore, in a first aspect of the present disclosure, there is provided a bispecific antibody. According to an embodiment of the present disclosure, the bispecific antibody includes a first antigen binding region, a second antigen binding region, and an Fc portion. The Fc portion includes a first Fc peptide fragment and a second Fc peptide fragment. The first antigen binding region includes a scFv fragment, and a binding protein of a first molecule or fragment thereof. The binding protein of the first molecule or fragment thereof is linked to an N-terminus of the scFv fragment. A C-terminus of the scFv fragment is linked to an N-terminus of the first Fc peptide fragment. The second antigen binding region includes a binding protein of a second molecule or fragment thereof. The binding protein of the second molecule or fragment thereof is linked to an N-terminus of the second Fc peptide fragment. The first molecule and the second molecule are PD-L1. The scFv fragment has CD3 binding activity.

The bispecific antibody according to embodiments of the present disclosure can simultaneously bind to CD3 and PD-L1, thereby promoting T cell activation and cytokine secretion. The bispecific antibody exhibits a stronger binding ability to tumor cell and a weaker binding ability to the T cell (with potentially higher safety), thereby being more conducive to distribution of a drug conjugated to the bispecific antibody into tumor tissue, increasing the effective concentration of the drug conjugated to the bispecific antibody in the tumor tissue, while reducing peripheral toxicity. Therefore, the bispecific antibody according to the embodiments of the present disclosure has high value in clinical application and drug development.

In a second aspect of the present disclosure, there is provided an isolated nucleic acid. According to an embodiment of the present disclosure, the isolated nucleic acid encodes the bispecific antibody according to the above. The isolated nucleic acid according to the embodiment of the present disclosure may encode a bispecific antibody capable of simultaneously targeting CD3 and PD-L1.

In a third aspect of the present disclosure, there is provided an expression vector. According to an embodiment of the present disclosure, the expression vector includes the isolated nucleic acid according to the above. The expression vector according to the embodiment of the present disclosure may effectively express the bispecific antibody according to the first aspect of the present disclosure.

In a fourth aspect of the present disclosure, there is provided a recombinant cell. According to an embodiment of the present disclosure, the recombinant cell carries the isolated nucleic acid according to the second aspect of the present disclosure, the expression vector according to the third aspect of the present disclosure, or the bispecific antibody according to the first aspect of the present disclosure. The recombinant cell according to the embodiment of the present disclosure is obtained by transfecting or transforming the expression vector according to the third aspect of the present disclosure and thus can efficiently express the bispecific antibody according to the first aspect of the present disclosure under suitable conditions.

In a fifth aspect of the present disclosure, there is provided a pharmaceutical composition. According to an embodiment of the present disclosure, the pharmaceutical composition includes: the bispecific antibody according to the first aspect of the present disclosure, the isolated nucleic acid according to the second aspect of the present disclosure, the expression vector according to the third aspect of the present disclosure, or the recombinant cell according to the fourth aspect of the present disclosure. As can be seen from the above, the bispecific antibody according to the first aspect can effectively promote the T cell to kill the tumor cell, providing better anticancer activity. In addition, the bispecific antibody according to the first aspect may be prepared using the above-mentioned isolated nucleic acid, expression vector, or recombinant cell. Therefore, the pharmaceutical composition including the above-mentioned bispecific antibody can further be used for preventing and/or treating a CD3-mediated and/or a PD-L1-mediated disease.

In a sixth aspect of the present disclosure, there is provided a kit. According to an embodiment of the present disclosure, the kit includes the bispecific antibody according to the first aspect of the present disclosure, the isolated nucleic acid according to the second aspect of the present disclosure, the expression vector according to the third aspect of the present disclosure, or the recombinant cell according to the fourth aspect of the present disclosure. The kit according to the embodiment of the present disclosure can effectively detect a CD3 protein and/or a PD-L1 protein.

In a seventh aspect of the present disclosure, there is provided use of the bispecific antibody according to the first aspect of the present disclosure, the isolated nucleic acid according to the second aspect of the present disclosure, the expression vector according to the third aspect of the present disclosure, or the recombinant cell according to the fourth aspect of the present disclosure in the manufacture of a kit. The kit is used for detecting a tumor target of CD3 and/or PD-L1.

In an eighth aspect of the present disclosure, there is provided use of the bispecific antibody according to the first aspect of the present disclosure, the isolated nucleic acid according to the second aspect of the present disclosure, the expression vector according to the third aspect of the present disclosure, the recombinant cell according to the fourth aspect of the present disclosure, or the pharmaceutical composition according to the fifth aspect of the present disclosure in the manufacture of a medicament, wherein the medicament is used for preventing and/or treating a CD3-mediated and/or a PD-L1-mediated disease. As can be seen from the above, the bispecific antibody according to the first aspect can effectively promote the T cell to kill the tumor cell and has better anticancer activity. In addition, the bispecific antibody according to the first aspect may be prepared using the above-mentioned isolated nucleic acid, expression vector, or recombinant cell. Therefore, a medicament manufactured using the above bispecific antibody can be used for preventing or treating the CD3-mediated and/or the PD-L1-mediated disease.

Additional aspects and advantages of the present disclosure will be provided at least in part in the following description or will become apparent at least in part from the following description or can be learned from practicing of the present disclosure.

### BRIEF DESCRIPTION OF THE DRAWINGS

The above and/or additional aspects and advantages of the present disclosure will become more apparent and more understandable from the following description of embodiments taken in conjunction with the accompanying drawings of which:

FIG. 1 is a schematic diagram of a CD3×PD-L1 bispecific antibody in Example 1 of the present disclosure.

FIG. 2 is a graph showing the ELISA results of the binding of a CD3×PD-L1 1:1 antibody and a CD3×PDL1 2:1 antibody to human CD3 protein in Example 2 of the present disclosure.

FIG. 3 is a graph showing the ELISA results of the binding of a CD3×PDL1 1:1 antibody and a CD3×PDL1 2:1 antibody to monkey CD3 protein in Example 2 of the present disclosure.

FIG. 4 is a graph showing the ELISA results of the binding of a CD3×PDL1 1:1 antibody and a CD3×PDL1 2:1 antibody to human PD-L1 protein in Example 2 of the present disclosure.

FIG. 5 is a graph showing the ELISA results of the binding of a CD3×PDL1 1:1 antibody and a CD3×PDL1 2:1 antibody to monkey PD-L1 protein in Example 2 of the present disclosure.

FIG. 6 is a graph showing the ELISA results of the bridging of CD3 and PDL1 by a CD3×PDL1 1:1 antibody and a CD3×PDL1 2:1 antibody in Example 3 of the present disclosure.

FIG. 7 is a graph showing the flow cytometry results of the binding of a CD3×PDL1 1:1 antibody and a CD3×PDL1 2:1 antibody to an A-375 human melanoma cell in Example 4 of the present disclosure.

FIG. 8 is a graph showing the flow cytometry results of the binding of a CD3×PDL1 1:1 antibody and a CD3×PDL1 2:1 antibody to an A549 human lung cancer cell in Example 4 of the present disclosure.

FIG. 9 is a graph showing the flow cytometry results of the binding of a CD3×PDL1 1:1 antibody and a CD3×PDL1 2:1 antibody to a CD8 T cell in Example 4 of the present disclosure.

FIG. 10 is a graph showing the results of a CD3×PDL1 1:1 antibody and a CD3×PDL1 2:1 antibody promoting PBMC to kill PANC-1 human pancreatic cancer in Example 5 of the present disclosure.

FIG. 11 is a graph showing the results of a CD3×PDL1 1:1 antibody and a CD3×PDL1 2:1 antibody promoting PBMC to kill A-375 human melanoma cell in Example 5 of the present disclosure.

FIG. 12 is a graph showing the results of a CD3×PDL1 1:1 antibody and a CD3×PDL1 2:1 antibody promoting PBMC to kill HPAFII human pancreatic cancer in Example 5 of the present disclosure.

FIG. 13 is a graph showing the results of a CD3×PDL1 1:1 antibody and a CD3×PDL1 2:1 antibody promoting PBMC to kill A549 human lung cancer cell in Example 5 of the present disclosure.

FIG. 14 is a graph showing the results of a CD3×PDL1 1:1 antibody and a CD3×PDL1 2:1 antibody promoting PBMC to kill PC-3 human prostate cancer cell in Example 5 of the present disclosure.

FIG. 15 is a graph showing the results of a CD3×PDL1 1:1 antibody and a CD3×PDL1 2:1 antibody promoting PBMC to kill HCT-15 human colorectal cancer cell in Example 5 of the present disclosure.

FIG. 16 is a graph showing the results of IL-2 secretion by PBMC promoted by a CD3×PDL1 2:1 antibody in Example 6 of the present disclosure.

FIG. 17 is a graph showing the results of a CD3×PDL1 2:1 antibody promoting the IFN-γ secretion by PBMC in Example 6 of the present disclosure.

FIG. 18 is a graph showing the in vivo efficacy results of a CD3×PDL1 2:1 antibody in an A-375 human melanoma mouse subcutaneous tumor-bearing model in Example 7 of the present disclosure.

### DETAILED DESCRIPTION

Embodiments of the present disclosure are described in detail below. The embodiments described below are illustrative and are intended to explain, rather than limiting the present disclosure.

It should be noted that the terms “first” and “second” are only used for descriptive purposes and cannot be understood as indicating or implying relative importance or implicitly indicating the number of technical features indicated. Therefore, the features defined as “first” and “second” may explicitly or implicitly include one or more of the features. Further, in the description of the present disclosure, unless otherwise specified, “plurality” means two or more.

The endpoints and any values of the ranges disclosed herein are not limited to the precise ranges or values. These ranges or values should be understood as including a value close to these ranges or values. For numerical ranges, each range between the endpoints and individual endpoints, and the individual points may be combined with each other to form one or more new numerical ranges, and such numerical ranges shall be considered as being specifically disclosed herein.

### TERMS AND DEFINITIONS

For a better understanding of the present disclosure, certain technical and scientific terms are specifically defined below. Unless otherwise clearly defined elsewhere in this document, all other technical and scientific terms used herein have the same meanings as commonly understood by those of ordinary skill in the art to which the present disclosure pertains. Abbreviations for amino acid residues are the standard 3-letter and/or 1-letter codes used in the art to refer to one of 20 common L-amino acids.

As used herein, a term “antibody” generally refers to an antibody that can recognize one or more antigenic epitopes, including but not limited to a monoclonal antibody, a polyclonal antibody, a dimer, a multimer, a multi-specific antibody (e.g., a bispecific antibody), a heavy-chain-only antibody, a triple-chain antibody, a single-chain Fv (scFv), a nanobody, and the like, and also includes an antibody fragment, as long as the antibody exhibits the desired biological activity (MiLLer et al. (2003) Jour. of ImmunoLogy 170:4854-4861). The antibody may be murine, human, humanized, chimeric, or from other species. The antibody may refer to a full-length heavy chain a full-length light chain, or an intact immunoglobulin molecule; or an immunologically active portion of any of one of these polypeptides, i.e., a molecule or a part thereof that contains an antigen-binding site that immunospecifically binds to a target antigen of interest, such targets include but not limited to the cancer cell or cells that produce an autoimmune antibody associated with an autoimmune disease.

In the present disclosure, in the variable region, the amino acid composition and arrangement order of some regions have a higher degree of variation, which is referred to as a “hypervariable region (HVR)”. The hypervariable region is a position where an antigen binds to the antibody and thus is also referred to as complementarity-determining region (CDR). There are three CDRs in both the heavy chain variable region and the light chain variable region.

In the present disclosure, the term “Fab fragment” generally refers to an antibody or a fragment that includes only a Fab molecule, which is composed of VH and CH1 of the heavy chain and an intact light chain. The light chain is linked to the heavy chain by a disulfide bond.

In the present disclosure, the term “F(ab′)₂ fragment” has two antigen-binding F(ab′) portions linked together by the disulfide bond.

In the present disclosure, the terms “single-chain antibody” and “scFv fragment” refer to antibodies or fragments formed by linking the heavy chain variable region and the light chain variable region of the antibody via a short peptide.

In the present disclosure, the term “bispecific antibody” is equivalent to “antibody fragment” or “antigen-binding antibody fragment”, and may include an intact antibody or a portion of the intact antibody, generally an antigen-binding region or a variable region, including but is not limited to: Fv, scFv, Fab, Fab′, F(ab′)₂, a scFv-Fc fragment, a linear antibody, or any fragment capable of increasing a half-life through chemical modification or incorporation into liposomes. Such chemical modification includes addition of poly(alkylene glycols), such as polyethylene glycol (“polyethylene glycolation, PEGylation”) (the PEGylated fragment referred to as Fv-PEG, scFv-PEG, Fab-PEG, F(ab′)₂-PEG or Fab′-PEG) (“PEG” means polyethylene glycol).

In the present disclosure, the term “expression vector” generally refers to a vector medium that carries a nucleic acid molecule capable of self-replication by insertion into a suitable host, which transfers the inserted nucleic acid molecule into and/or between host cells. The expression vector may include a vector primarily used for inserting DNA or RNA into cells, a vector primarily used for replicating DNA or RNA, and a vector primarily used for transcription and/or translation of DNA or RNA. The expression vector also includes a vector having multiple functions as described above. The expression vector may be a polynucleotide that can be transcribed and translated into a polypeptide when introduced into a suitable host cell. Generally, by culturing a suitable host cell including the vector, the expression vector may produce a desired expression product.

In the present disclosure, the term “pharmaceutical composition” generally refers to a substance present in a unit dosage form and can be prepared by methods commonly used in the pharmaceutical field. These methods include the step of combining the active ingredient with the carrier which constitutes one or more accessory ingredients. Typically, the method of preparing the pharmaceutical composition is to thoroughly mix the active bispecific antibody with the liquid carrier to ensure that they are uniformly bound together.

In the present disclosure, the term “pharmaceutically acceptable” refers to any ingredient that is suitable for use in humans and/or mammals without excessive adverse side effects (such as toxicity, irritation, and allergic reactions), i.e., a substance with at a reasonable benefit/risk ratio.

In the present disclosure, the term “pharmaceutically acceptable excipient” may include any solvent, solid excipient, diluent, or other liquid excipients, etc., suitable for a specific target dosage form. Except for any conventional excipient that is incompatible with a compound of the present disclosure, for example, which produces any adverse biological effects or interact in a deleterious manner with any other components of a pharmaceutically acceptable composition, their use is also considered within the scope of the present disclosure.

In the present disclosure, the term “administration” refers to introducing a predetermined amount of a substance into a patient by a suitable route. The bispecific antibody or the pharmaceutical composition of the present disclosure may be administered by any common route, as long as the bispecific antibody or the pharmaceutical composition can reach the intended tissue. Various modes of administration may be contemplated, including peritoneal, intravenous injection, intramuscular injection, subcutaneous injection, and the like, but the present disclosure is not limited to these exemplified modes of administration. The pharmaceutical composition of the present disclosure is administered by the intravenous injection or the subcutaneous injection.

In the present disclosure, the term “treatment” refers to obtaining a desired pharmacological and/or physiological effect. The effect may be prophylactic in terms of completely or partially preventing a disease or its symptom, and/or therapeutic in terms of partially or completely curing the disease or its adverse effects. As used herein, the “treatment” encompasses diseases in mammals, particularly in humans, and includes: (a) preventing an occurrence of a disease or a condition in an individual who is susceptible to the disease but has not yet been diagnosed with the disease; (b) inhibiting the disease, such as arresting the progression of the disease; or (c) alleviating the disease, such as alleviating the symptoms associated with the disease. As used herein, the “treatment” encompasses any administration of a medicament to an individual to treat, cure, alleviate, ameliorate, lessen, or inhibit the disease in the individual, including but not limited to administering the medicament described herein to an individual in need thereof.

In the present disclosure, the term “effective amount” or “effective dose” refers to an amount that can produce a function or activity in humans and/or animals and is acceptable to humans and/or animals.

The present disclosure provides a bispecific antibody targeting CD3 and PD-L1, an isolated nucleic acid, an expression vector, a recombinant cell, a pharmaceutical composition, a kit, and use thereof, which will be described in detail below.

### Bispecific Antibody

In a first aspect of the present disclosure, there is provided a bispecific antibody. According to an embodiment of the present disclosure, the bispecific antibody includes a first antigen binding region, a second antigen binding region, and an Fc portion. The Fc portion includes a first Fc peptide fragment and a second Fc peptide fragment. The first antigen binding region includes a scFv fragment, and a binding protein of a first molecule or fragment thereof. The binding protein of the first molecule or fragment thereof is linked to an N-terminus of the scFv fragment. A C-terminus of the scFv fragment is linked to an N-terminus of the first Fc peptide fragment. The second antigen binding region includes a binding protein of a second molecule or fragment thereof. The binding protein of the second molecule or fragment thereof is linked to an N-terminus of the second Fc peptide fragment. The first molecule and the second molecule are PD-L1. The scFv fragment has CD3 binding activity. The bispecific antibody according to the embodiments of the present disclosure can simultaneously bind to CD3 on a surface of the T cell and PD-L1 on a surface of the tumor cell, thereby promoting T cell activation and cytokine secretion. The bispecific antibody exhibits a stronger binding ability to tumor cell and a weaker binding ability to the T cell (which can help reduce non-specific activation and a potential toxic reaction in a normal tissue, and improve safety), thereby being more conducive to distribution of a drug (a small-molecule drug, a targeted drug, a radionuclide, a cytotoxin, an immunomodulator, etc.) conjugated to the bispecific antibody into tumor tissue, increasing the effective concentration of the drug conjugated to the bispecific antibody in the tumor tissue, while reducing peripheral toxicity. Therefore, the bispecific antibody according to the embodiments of the present disclosure has high value in clinical application and drug development.

According to the embodiments of the present disclosure, the above bispecific antibody may further include at least one of the following additional technical features.

According to the embodiments of the present disclosure, the binding protein or fragment thereof is selected from at least one of an antibody or antigen-binding fragment thereof, a receptor, and a ligand.

According to the embodiments of the present disclosure, the binding protein of the first molecule or fragment thereof and/or the second molecule or fragment thereof are each independently selected from at least one of a Fab fragment or an F(ab′)₂ fragment.

According to the embodiments of the present disclosure, the binding protein of the first molecule or fragment thereof is a first Fab fragment; and/or the binding protein of the second molecule or fragment thereof is a second Fab fragment.

According to the embodiments of the present disclosure, each of the first Fab fragment and the second Fab fragment includes: a first heavy chain variable region CDR1, the amino acid sequence of the first heavy chain variable region CDR1 being set forth in SEQ ID NO: 1; a first heavy chain variable region CDR2, the amino acid sequence of the first heavy chain variable region CDR2 being set forth in SEQ ID NO: 2; a first heavy chain variable region CDR3, the amino acid sequence of the first heavy chain variable region CDR3 being set forth in SEQ ID NO: 3; a first light chain variable region CDR1, the amino acid sequence of the first light chain variable region CDR1 being set forth in SEQ ID NO: 4; a first light chain variable region CDR2, the amino acid sequence of the first light chain variable region CDR2 being set forth in SEQ ID NO: 5; and a first light chain variable region CDR3, the amino acid sequence of the first light chain variable region CDR3 being set forth in SEQ ID NO: 6.

According to the embodiments of the present disclosure, the first Fab fragment and/or the second Fab fragment includes a first heavy chain framework region and/or a first light chain framework region.

According to the embodiments of the present disclosure, at least part of the first heavy chain framework region and/or the first light chain framework region is from at least one of a murine antibody, a human antibody, a primate antibody, a bovine antibody, an equine antibody, a dairy cow antibody, a porcine antibody, an ovine antibody, a caprine antibody, a canine antibody, a feline antibody, a rabbit antibody, a camel antibody, an donkey antibody, a deer antibody, a mink antibody, a chicken antibody, a duck antibody, a goose antibody, a turkey antibody, a gamecock antibody, or a mutant thereof, and preferably, from at least one of the murine antibody, the human antibody, or the primate antibody.

According to the embodiments of the present disclosure, the first heavy chain variable regions of the first Fab fragment and the second Fab fragment are each independently selected from an amino acid sequence as set forth in SEQ ID NO: 7 or an amino acid sequence having at least 90% identity thereto; and/or the first light chain variable regions of the first Fab fragment and the second Fab fragment are each independently selected from an amino acid sequence as set forth in SEQ ID NO: 8 or an amino acid sequence having at least 90% identity thereto.

According to the embodiments of the present disclosure, the first Fab fragment and/or the second Fab fragment further includes a CH1 fragment and a CL fragment. A C-terminus of the first heavy chain variable region is linked to an N-terminus of the CH1 fragment. A C-terminus of the first light chain variable region is linked to an N-terminus of the CL fragment. The CH1 fragment is linked to the CL fragment via an interchain disulfide bond.

According to the embodiments of the present disclosure, the CH1 fragment has an amino acid sequence as set forth in SEQ ID NO: 25; and/or the CL fragment has an amino acid sequence as set forth in SEQ ID NO: 26.

According to the embodiments of the present disclosure, a C-terminus of the CH1 fragment of the first Fab fragment is linked to the N-terminus of the scFv fragment, and the C-terminus of the scFv fragment is linked to the N-terminus of the first Fc peptide fragment; and/or a C-terminus of the CH1 fragment of the second Fab fragment is linked to the N-terminus of the second Fc peptide fragment.

According to the embodiments of the present disclosure, each of the first Fab fragment and the second Fab fragment includes: a first peptide fragment having an amino acid sequence as set forth in SEQ ID NO: 27; and a second peptide fragment having an amino acid sequence as set forth in SEQ ID NO: 28.

According to the embodiments of the present disclosure, the scFv fragment includes: a second heavy chain variable region CDR1, the amino acid sequence of the second heavy chain variable region CDR1 being set forth in SEQ ID NO: 9; a second heavy chain variable region CDR2, the amino acid sequence of the second heavy chain variable region CDR2 being set forth in SEQ ID NO: 10; a second heavy chain variable region CDR3, the amino acid sequence of the second heavy chain variable region CDR3 being set forth in SEQ ID NO: 11; a second light chain variable region CDR1, the amino acid sequence of the second light chain variable region CDR1 being set forth in SEQ ID NO: 12; a second light chain variable region CDR2, the amino acid sequence of the second light chain variable region CDR2 being set forth in SEQ ID NO: 13; and a second light chain variable region CDR3, the amino acid sequence of the second light chain variable region CDR3 being set forth in SEQ ID NO: 14. Therefore, when the scFv fragment of the bispecific antibody of the present disclosure has the above-described CDR amino acid sequences, CD3 binding activity can be further reduced, a tumor cell binding ability can be enhanced, and the activity of promoting T cells to kill the tumor cells can be further improved.

According to the embodiments of the present disclosure, the scFv fragment includes a second heavy chain framework region and/or a second light chain framework region.

According to the embodiments of the present disclosure, at least part of the second heavy chain framework region and/or the second light chain framework region is from at least one of a murine antibody, a human antibody, a primate antibody, a bovine antibody, an equine antibody, a dairy cow antibody, a porcine antibody, an ovine antibody, a caprine antibody, a canine antibody, a feline antibody, a rabbit antibody, a camel antibody, an donkey antibody, a deer antibody, a mink antibody, a chicken antibody, a duck antibody, a goose antibody, a turkey antibody, a gamecock antibody, or a mutant thereof, and preferably, from at least one of the murine antibody, the human antibody, or the primate antibody.

It should be noted that an immunoglobulin described herein may be one of any type (e.g., IgG, IgE, IgM, IgD, and IgA), class (e.g., IgG1, IgG2, IgG3, IgG4, IgA1, and IgA2), or subclass, including engineered subclass with altered Fc portion that provides reduced or enhanced effector cell activity. The immunoglobulin may be derived from any species.

According to the embodiments of the present disclosure, the scFv fragment includes: a second heavy chain variable region having an amino acid sequence as set forth in SEQ ID NO: 15 or an amino acid sequence having at least 90% identity thereto; and a second light chain variable region having an amino acid sequence as set forth in SEQ ID NO: 16 or an amino acid sequence having at least 90% identity thereto.

According to the embodiments of the present disclosure, the scFv fragment further includes a linker peptide 1. An N-terminus of the linker peptide 1 is linked to a C-terminus of the second heavy chain variable region. A C-terminus of the linker peptide 1 is linked to an N-terminus of the second light chain variable region. Alternatively, the N-terminus of the linker peptide 1 is linked to a C-terminus of the second light chain variable region, and the C-terminus of the linker peptide 1 is linked to an N-terminus of the second heavy chain variable region.

According to the embodiments of the present disclosure, the linker peptide 1 has an amino acid sequence as set forth in (GGGGS)ₙ, where n is an integer greater than or equal to 1, and preferably, 1, 2, 3, 4, 5, 6, 7, 8, 9, or 10.

According to the embodiments of the present disclosure, the scFv fragment has an amino acid sequence as set forth in SEQ ID NO: 17.

According to the embodiments of the present disclosure, the linker peptide 1 has an amino acid sequence as set forth in SEQ ID NO: 18.

According to the embodiments of the present disclosure, the first antigen binding region further includes a linker peptide 2. Wherein, a C-terminus of the CH1 fragment of the first Fab fragment is linked to an N-terminus of the linker peptide 2, and a C-terminus of the linker peptide 2 is linked to the N-terminus of the scFv fragment.

According to the embodiments of the present disclosure, the linker peptide 2 has an amino acid sequence as set forth in (GGGGS)ₙ, where n is an integer greater than or equal to 1.

According to the embodiments of the present disclosure, the linker peptide 2 has an amino acid sequence as set forth in (GGGGS)ₙ, where n is 1, 2, 3, 4, 5, 6, 7, 8, 9, or 10.

According to the embodiments of the present disclosure, the linker peptide 2 has an amino acid sequence as set forth in SEQ ID NO: 19.

According to the embodiments of the present disclosure, the first Fc peptide fragment and the second Fc peptide fragment are linked via a knob-into-hole structure.

According to the embodiments of the present disclosure, each of the first Fc peptide fragment and the second Fc peptide fragment is a human Fc peptide fragment.

According to the embodiments of the present disclosure, the human Fc peptide fragment is a human IgG1 Fc peptide fragment.

According to the embodiments of the present disclosure, the first Fc peptide fragment has an amino acid sequence as set forth in SEQ ID NO: 20, the second Fc peptide fragment has an amino acid sequence as set forth in SEQ ID NO: 21.

According to the embodiments of the present disclosure, the bispecific antibody includes: a first polypeptide chain having an amino acid sequence as set forth in SEQ ID NO: 22 or an amino acid sequence having at least 80% identity thereto; a second polypeptide chain having an amino acid sequence as set forth in SEQ ID NO: 23 or an amino acid sequence having at least 80% identity thereto; a third polypeptide chain having an amino acid sequence as set forth in SEQ ID NO: 24 or an amino acid sequence having at least 80% identity thereto; and a fourth polypeptide chain having the amino acid sequence as set forth in SEQ ID NO: 24 or the amino acid sequence having at least 80% identity thereto.

It should be noted that, without substantially affecting the CD3 and PD-L1 binding activity of a multispecific antibody (retaining at least 95% of the activity), those skilled in the art may substitute, add, and/or delete one or more (e.g., 1, 2, 3, 4, 5, 6, 7, 8, 9, or 10 or more) amino acids in the amino acid sequence of a polypeptide chain included in the multispecific antibody of the present disclosure, to obtain variants of the amino acid sequence of a polypeptide chain of the bispecific antibody, which are considered to be included within the scope of protection of the present disclosure. For example, amino acids with similar properties are substituted in the polypeptide chain. An amino acid sequence of the above variants of the present disclosure may have at least 80%, 85%, 90%, 95%, 96%, 97%, 98%, or 99% identity (or homology) to a reference amino acid sequence.

According to the embodiments of the present disclosure, the bispecific antibody includes: a first polypeptide chain having an amino acid sequence as set forth in SEQ ID NO: 22; a second polypeptide chain having an amino acid sequence as set forth in SEQ ID NO: 23; a third polypeptide chain having an amino acid sequence as set forth in SEQ ID NO: 24; and a fourth polypeptide chain having an amino acid sequence as set forth in SEQ ID NO: 24. This bispecific antibody has higher tumor cell binding capacity, lower T cell binding capacity, stronger tumor cell killing ability and higher safety. Notably, it was unexpectedly found that while reducing the T cell binding capacity, the bispecific antibody of the present disclosure can further enhance the killing ability on the tumor cells and has excellent anti-tumor activity and high value in clinical application and drug development.

### Isolated Nucleic Acid

In a second aspect of the present disclosure, there is provided an isolated nucleic acid. According to an embodiment of the present disclosure, the isolated nucleic acid encodes the bispecific antibody according to the above. The isolated nucleic acid according to the embodiment of the present disclosure may encode a bispecific antibody capable of simultaneously targeting CD3 and PDL1.

It should be noted that with regard to the nucleic acid molecule mentioned herein, it should be understood by those skilled in the art that the nucleic acid molecule actually includes any one or both of complementary double strands. For the sake of convenience, although only one strand is shown in most cases herein, the other strand complementary thereof is also disclosed in essence. In addition, if the amino acid sequence of the molecule of the present disclosure, including for example the DNA form or the RNA form, discloses one form, meaning the other form is also disclosed.

It should be appreciated by those skilled in the art that the foregoing features and advantages of the bispecific antibody are equally applicable to the isolated nucleic acid, which will not be repeated herein.

### Expression Vector

In a third aspect of the present disclosure, there is provided an expression vector. According to an embodiment of the present disclosure, the expression vector includes the isolated nucleic acid according to the above. The expression vector according to the embodiment of the present disclosure may effectively express the bispecific antibody according to the first aspect of the present disclosure.

When the above-described isolated nucleic acid is linked to the expression vector, the isolated nucleic acid may be directly or indirectly linked to control elements on the expression vector, as long as these control elements can control the translation and expression of the isolated nucleic acid. Of course, these control elements may be derived directly from the expression vector itself or may be exogenous, that is, not derived from the expression vector itself. It is sufficient that the isolated nucleic acid is operably linked to the control elements.

According to the embodiments of the present disclosure, the expression vector may be obtained by operably linking the isolated nucleic acid to a commercially available expression vector (such as a plasmid or a viral vector). The expression vector herein is not particularly limited. A commonly used plasmid may be used, such as pSeTag2, PEE14, and pMH3.

In the present disclosure, the term “operably linked” means that the exogenous gene is linked to the vector, so that the control element inside the vector, such as amino acid sequences for transcriptional control and amino acid sequences for translational control, can play their intended function of regulating the transcription and translation of the exogenous gene. Commonly used vector, for example, may be a viral vector, the plasmid, a bacteriophage and the like. After the expression vector according to some specific embodiments of the present disclosure is introduced into the suitable recipient cell, the expression of the aforementioned isolated nucleic acid can be effectively achieved under the mediation of a regulatory system, thereby obtaining a large-scale in vitro production of the protein encoded by the isolated nucleic acid.

According to the embodiments of the present disclosure, the above expression vector may further include at least one of the following additional technical features.

According to the embodiments of the present disclosure, the expression vector is a eukaryotic vector or a prokaryotic vector.

According to the embodiments of the present disclosure, the expression vector includes at least one selected from a plasmid vector, an adenovirus vector, a lentiviral vector, and an adeno-associated virus vector.

It should be appreciated by those skilled in the art that the foregoing features and advantages of the bispecific antibody are equally applicable to the expression vector, which will not be repeated herein.

### Recombinant Cell

In a fourth aspect of the present disclosure, there is provided a recombinant cell. According to an embodiment of the present disclosure, the recombinant cell carries the isolated nucleic acid according to the second aspect of the present disclosure, the expression vector according to the third aspect of the present disclosure or expresses the bispecific antibody according to the first aspect of the present disclosure. The recombinant cell according to the embodiment of the present disclosure is obtained by transfecting or transforming the expression vector according to the third aspect of the present disclosure and thus can efficiently express the bispecific antibody according to the first aspect of the present disclosure under suitable conditions.

According to the embodiments of the present disclosure, the above recombinant cell may further include at least one of the following additional technical features.

According to the embodiments of the present disclosure, the recombinant cell is a prokaryotic cell, a eukaryotic cell, or a bacteriophage.

According to the embodiments of the present disclosure, the prokaryotic cell is Escherichia coli, Bacillus subtilis, Streptomyces, or Proteus mirabilis.

According to the embodiments of the present disclosure, the eukaryotic cell is a fungus, an insect cell, a plant cell, or a mammalian cell.

According to the embodiments of the present disclosure, the fungus is Pichia pastoris, Saccharomyces cerevisiae, Schizosaccharomyces pombe, or Trichoderma.

According to the embodiments of the present disclosure, the insect cell is a Spodoptera frugiperda cell. According to the embodiments of the present disclosure, the plant cell is a tobacco plant cell. According to the embodiments of the present disclosure, the mammalian cell is a BHK cell, a CHO cell, a COS cell, a myeloma cell, or a human embryonic kidney 293 cell, and does not include an animal germ cell, a fertilized egg, or an embryonic stem cell.

According to the embodiments of the present disclosure, the recombinant cell is the mammalian cell.

According to the embodiments of the present disclosure, the recombinant cell is the BHK cell, the CHO cell, the COS cell, or an NSO cell.

It should be noted that the “suitable conditions” described in the specification of the present disclosure refer to conditions suitable for the expression of the bispecific antibody described in the present disclosure. It is readily understood by those skilled in the art that the conditions suitable for the expression of the bispecific antibody include, but are not limited to, suitable transformation or transfection methods, suitable transformation or transfection conditions, healthy host cell status, suitable host cell density, suitable cell culture environment, and suitable cell culture time. The “suitable conditions” are not particularly limited, and those skilled in the art can optimize the most suitable conditions for the expression of the bispecific antibody according to a specific environment of the laboratory.

It should be appreciated by those skilled in the art that the foregoing features and advantages of the bispecific antibody are equally applicable to the recombinant cell, which will not be repeated herein.

### Pharmaceutical Composition

In a fifth aspect of the present disclosure, there is provided a pharmaceutical composition. According to an embodiment of the present disclosure, the pharmaceutical composition includes: the bispecific antibody according to the first aspect of the present disclosure, the isolated nucleic acid according to the second aspect of the present disclosure, the expression vector according to the third aspect of the present disclosure, or the recombinant cell according to the fourth aspect of the present disclosure. According to the embodiments of the present disclosure, the bispecific antibody can effectively promote PBMC to kill the tumor cells, has better anticancer activity, and has high value of clinical application and drug development. Therefore, the pharmaceutical composition including the above-mentioned bispecific antibody can further be used for preventing and/or treating CD3-mediated and/or a PD-L1-mediated diseases.

According to the embodiments of the present disclosure, the above pharmaceutical composition may further include at least one of the following additional technical features.

According to the embodiments of the present disclosure, the pharmaceutical composition further includes a pharmaceutically acceptable excipient.

According to the embodiments of the present disclosure, the pharmaceutical composition is an injection.

It should be noted that the pharmaceutical composition includes combinations separated in time and/or space, as long as the combinations can act together to achieve the purpose of the present disclosure. For example, the component contained in the pharmaceutical composition may be administered to a subject as a whole, or separately. When the components contained in the pharmaceutical composition are administered to the subject separately, each component may be administered to the subject simultaneously or sequentially.

The pharmaceutical composition of the present disclosure contains a safe and effective amount of an active ingredient of the present disclosure and the pharmaceutically acceptable excipients. Such excipients include, but are not limited to, saline, a buffer, glucose, water, glycerol, ethanol, and a combination thereof. Generally, the preparation of the pharmaceutical composition should be compatible with the mode of administration. A dosage form of the pharmaceutical composition of the present disclosure is the injection. For example, the pharmaceutical composition is prepared by a conventional methods using physiological saline or an aqueous solution containing glucose and other adjuvants. The pharmaceutical composition is preferably manufactured under sterile conditions.

An effective amount of the bispecific antibody described in the present disclosure may vary depending on the mode of administration, and the severity of the disease to be treated. Selection of a preferred effective amount may be determined by those skilled in the art based on various factors (e.g., via clinical trials). The factors include, but are not limited to a pharmacokinetic parameter of the active ingredient, such as bioavailability, metabolism, half-life, etc., the severity of the disease to be treated in the patient, a body weight of the patient, an immune status of the patient, route of administration, and the like. For example, several divided doses may be administered daily, or the dose may be proportionally reduced depending on exigencies of the treatment situation.

The pharmaceutically acceptable excipients described in the present disclosure include, but are not limited to water, saline, liposomes, lipids, proteins, protein-antibody conjugates, peptides, cellulose, nanogels, or a combination thereof. The selection of the carrier needs to be compatible with the mode of administration, which is well known to those skilled in the art.

It should be appreciated by those skilled in the art that the foregoing features and advantages of the bispecific antibody are equally applicable to the pharmaceutical composition, which will not be repeated herein.

### Kit

In a sixth aspect of the present disclosure, there is provided a kit. According to an embodiment of the present disclosure, the kit includes the bispecific antibody according to the first aspect of the present disclosure, the isolated nucleic acid according to the second aspect of the present disclosure, the expression vector according to the third aspect of the present disclosure, or the recombinant cell according to the fourth aspect of the present disclosure.

As mentioned above, the bispecific antibody of the embodiments of the present disclosure can specifically bind to CD3 and PD-L1. The kit related to a CD3 protein and/or a PD-L1 protein developed by utilizing the characteristics may be applied to a related research on the CD3 protein and/or the PD-L1 protein, such as detection and/or enrichment, and/or separation and purification of the CD3 protein and/or the PD-L1 protein of human beings or other mammals.

The kit can effectively detect, enrich, or separate and purify the CD3 protein and/or the PD-L1 protein in biological samples, and thus further used in scientific research such as qualitatively or quantitatively detecting CD3 protein molecules and/or a PD-L1 protein molecules in biological samples. More specifically, the kit may be used in kits for immunoblotting, immunoprecipitation, and the like that involve detection using the specific binding properties of the CD3 protein and/or the PD-L1 protein and the antibody. The kit may include any one or more of the following: antagonists, the bispecific antibody of the present disclosure or medicament reference materials, protein purification columns, an immunoglobulin affinity purification buffers, or cell assay diluents. The bispecific antibody of the present disclosure may be used in various types of diagnostic tests, for example, in vitro or in vivo detection of the presence of various diseases or medicaments, toxins, or other proteins. For example, the bispecific antibody may be used to detect CD3-mediated and/or a PD-L1-mediated disease by testing the serum or blood of the subject.

It should be appreciated by those skilled in the art that the foregoing features and advantages of the bispecific antibody are equally applicable to the kit, which will not be repeated herein.

### Use in the Manufacture of a Kit

In a seventh aspect of the present disclosure, there is provided use of the bispecific antibody according to the first aspect of the present disclosure, the isolated nucleic acid according to the second aspect of the present disclosure, the expression vector according to the third aspect of the present disclosure, or the recombinant cell according to the fourth aspect of the present disclosure in the manufacture of a kit. The kit is used for detecting a tumor target of CD3 and/or PD-L1.

According to the embodiments of the present disclosure, the tumor target is PD-L1.

According to the embodiments of the present disclosure, a PD-L1-mediated disease includes a cancer or transplant rejection, an autoimmune disease, and an infectious disease.

According to the embodiments of the present disclosure, the cancer includes at least one of lung cancer, liver cancer, ovarian cancer, cervical cancer, skin cancer, bladder cancer, colon cancer, breast cancer, glioma, renal cancer, gastric cancer, esophageal cancer, oral squamous cell carcinoma, and head and neck cancer.

As mentioned above, the bispecific antibody of the embodiments of the present disclosure can specifically bind to CD3 and PD-L1. Therefore, the bispecific antibody can be used for detecting CD3 and/or PD-L1. Further, the bispecific antibody can be used in the manufacture of a kit related to CD3 and/or PD-L1 and be applied to the scientific research, such as qualitatively or quantitatively detecting CD3 protein molecules and/or PD-L1 protein molecules i biological samples. More specifically, the bispecific antibody may be used in kits for immunoblotting, immunoprecipitation, and the like that involve detection using the specific binding properties of the CD3 protein and/or the PD-L1 protein and the antibody. The kit may include any one or more of the following: antagonists, the bispecific antibody of the present disclosure or the medicament reference materials, protein purification columns, immunoglobulin affinity purification buffers, or cell assay diluents. The bispecific antibody of the present disclosure may be used in various types of diagnostic tests, for example, in vitro or in vivo detection of the presence of various diseases or medicaments, toxins, or other proteins. For example, the bispecific antibody may be used to detect the CD3 and/or PD-L1 by testing the serum or blood of the subject.

It should be appreciated by those skilled in the art that the foregoing features and advantages of the bispecific antibody are equally applicable to the use, which will not be repeated herein.

### Use in the Manufacture of Medicament

In an eighth aspect of the present disclosure, there is provided use of the bispecific antibody according to the first aspect of the present disclosure, the isolated nucleic acid according to the second aspect of the present disclosure, the expression vector according to the third aspect of the present disclosure, the recombinant cell according to the fourth aspect of the present disclosure, or the pharmaceutical composition according to the fifth aspect of the present disclosure in the manufacture of a medicament. The medicament is used for preventing and/or treating a CD3-mediated and/or a PD-L1-mediated disease.

According to the embodiments of the present disclosure, the above use may further include at least one of the following additional technical features.

According to the embodiments of the present disclosure, the CD3-mediated disease includes an autoimmune disease.

According to the embodiments of the present disclosure, the autoimmune disease includes at least one of systemic lupus erythematosus, rheumatoid arthritis, systemic vasculitis, scleroderma, dermatomyositis, autoimmune hemolytic anemia, thyroid autoimmune disease, ulcerative colitis, chronic lymphocytic thyroiditis, hyperthyroidism, insulin-dependent diabetes mellitus, myasthenia gravis, pernicious anemia with chronic atrophic gastritis, pulmonary hemorrhage nephritis syndrome, pemphigus vulgaris, pemphigoid, primary biliary cirrhosis, multiple sclerosis, and acute idiopathic polyneuritis.

It should be appreciated by those skilled in the art that the foregoing features and advantages of the bispecific antibody are equally applicable to the use, which will not be repeated herein.

### Method for Treating a Disease

In a ninth aspect of the present disclosure, there is provided a method for preventing and/or treating CD3-mediated and/or PDL1-mediated diseases. According to the embodiments of the present disclosure, the method includes administering a pharmaceutically acceptable dosage of the aforementioned bispecific antibody, the aforementioned isolated nucleic acid, the aforementioned expression vector, the aforementioned recombinant cell, or the aforementioned pharmaceutical composition to the subject.

It should be noted that the terms “subject”, “individual,” and “patient” may be used interchangeably herein and refer to a mammal that is evaluated for treatment and/or is treated. In one embodiment, the mammal is human. The terms “subject”, “individual,” and “patient” include but are not limited to individuals with cancer, individuals with autoimmune disease, individuals with pathogen infection, and the like. The subject may be human, and also includes other mammals, especially mammals serving as laboratory models for human disease, such as mouse or rat.

The effective amount of the bispecific antibody, the isolated nucleic acid, the expression vector, the recombinant cell, or the pharmaceutical composition described in the present disclosure may vary depending on the mode of administration, and the severity of the disease to be treated. The selection of the preferred effective amount may be determined by those skilled in the art based on various factors (e.g., via the clinical trial). The factors include, but are not limited to pharmacokinetic parameters of the active ingredient, such as bioavailability, metabolism, half-life, etc., the severity of the disease to be treated in the patient, the body weight of the patient, the immune status of the patient, the route of administration, and the like. For example, several divided doses may be administered daily, or the dose may be proportionally reduced depending on the exigencies of the treatment situation.

According to the embodiments of the present disclosure, the above method may further include at least one of the following additional technical features.

According to the embodiments of the present disclosure, the CD3-mediated disease includes an autoimmune disease.

According to the embodiments of the present disclosure, the autoimmune disease includes at least one of systemic lupus erythematosus, rheumatoid arthritis, systemic vasculitis, scleroderma, dermatomyositis, autoimmune hemolytic anemia, thyroid autoimmune disease, ulcerative colitis, chronic lymphocytic thyroiditis, hyperthyroidism, insulin-dependent diabetes mellitus, myasthenia gravis, pernicious anemia with chronic atrophic gastritis, pulmonary hemorrhage nephritis syndrome, pemphigus vulgaris, pemphigoid, primary biliary cirrhosis, multiple sclerosis, and acute idiopathic polyneuritis.

According to the embodiments of the present disclosure, the PD-L1-mediated disease is cancer, a disease caused by transplant rejection, the autoimmune disease, or the infectious disease.

According to the embodiments of the present disclosure, the cancer is at least one of lung cancer, liver cancer, ovarian cancer, cervical cancer, skin cancer, bladder cancer, colon cancer, breast cancer, glioma, renal cancer, gastric cancer, esophageal cancer, oral squamous cell carcinoma, and head and neck cancer.

It should be appreciated by those skilled in the art that the foregoing features and advantages of the bispecific antibody are equally applicable to the method, which will not be repeated herein.

The amino acid sequences involved in the present disclosure are detailed in Table 1.

TABLE 1
Description of Amino Acid Sequences

| SEQ | | |
|---|---|---|
| ID NO: | Sequence | Description |
| 1 | GYSFTGYY | PDL1 HCDR1 |
| 2 | INPYNGAT | PDL1 HCDR2 |
| 3 | VPTGMGYFLMDY | PDL1 HCDR3 |
| 4 | QDVGSA | PDL1 LCDR1 |
| 5 | WAS | PDL1 LCDR2 |
| 6 | QQFTSYPT | PDL1 LCDR3 |
| 7 | QVQLVQSGAEVKKPGASVKVSCKASGYSFTGYYMHWVRQAPGQGLEWIGRINPYN | Heavy chain |
| | GATTYSQNFKDRATMTVDTSISTAYMELSRLRSDDTAVYYCVPTGMGYFLMDYWG | variable |
| | QGTLVTVSS | region of PDL1 |
| 8 | DIQLTQSPSFLSASVGDRVTITCKASQDVGSAVVWYQQKPGKAPKLLIYWASTRHTG | Light chain |
| | VPSRFSGSGSGTEFTLTISSLQPEDFATYFCQQFTSYPTFGQGTKLEIK | variable |
| | | region of PDL1 |
| 9 | GFTFNTYA | CD3 HCDR1 |
| 10 | IRSKYNNYAT | CD3 HCDR2 |
| 11 | VRHGNFGNSYVSWFAY | CD3 HCDR3 |
| 12 | TGAVTTSNY | CD3 LCDR1 |
| 13 | GTN | CD3 LCDR2 |
| 14 | ALWYSNLWV | CD3 LCDR3 |
| 15 | EVQLLESGGGLVQPGGSLKLSCAASGFTFNTYAMNWVRQAPGKGLEWVARIRSKY | Heavy chain |
| | NNYATYYADSVKDRFTISRDDSKNTAYLQMNNLKTEDTAVYYCVRHGNFGNSYVS | variable region |
| | WFAYWGQGTLVTVSS | of CD3 |
| 16 | ELVVTQEPSLTVSPGGTVTLTCRSSTGAVTTSNYANWVQQKPGQAPRGLIGGTNKRA | Light chain |
| | PGTPARFSGSLLGGKAALTLSGVQPEDEAEYYCALWYSNLWVFGGGTKLTVL | variable region |
| | | of CD3 |
| 17 | EVQLLESGGGLVQPGGSLKLSCAASGFTFNTYAMNWVRQAPGKGLEWVARIRSKY | CD3 ScFv |
| | NNYATYYADSVKDRFTISRDDSKNTAYLQMNNLKTEDTAVYYCVRHGNFGNSYVS | fragment |
| | WFAYWGQGTLVTVSSGGGGSGGGGSGGGGSELVVTQEPSLTVSPGGTVTLTCRSST | |
| | GAVTTSNYANWVQQKPGQAPRGLIGGTNKRAPGTPARFSGSLLGGKAALTLSGVQP | |
| | EDEAEYYCALWYSNLWVFGGGTKLTVL | |
| 18 | GGGGSGGGGSGGGGS | Linker peptide 1 |
| 19 | GGGGS | Linker peptide 2 |
| 20 | PKSSDKTHTCPPCPAPEAAGGPSVFLFPPKPKDTLMISRTPEVTCVVVDVSHEDPEVK | First Fc peptide |
| | FNWYVDGVEVHNAKTKPREEQYNSTYRVVSVLTVLHQDWLNGKEYKCKVSNKAL | fragment |
| | PAPIEKTISKAKGQPREPQVYTLPPCREEMTKNQVSLWCLVKGFYPSDIAVEWESNG | |
| | QPENNYKTTPPVLDSDGSFFLYSKLTVDKSRWQQGNVFSCSVMHEALHNHYTQKSL | |
| | SLSP | |
| 21 | PKSCDKTHTCPPCPAPEAAGGPSVFLFPPKPKDTLMISRTPEVTCVVVDVSHEDPEVK | Second Fc |
| | FNWYVDGVEVHNAKTKPREEQYNSTYRVVSVLTVLHQDWLNGKEYKCKVSNKAL | peptide fragment |
| | PAPIEKTISKAKGQPREPQVCTLPPSREEMTKNQVSLSCAVKGFYPSDIAVEWESNGQ | |
| | PENNYKTTPPVLDSDGSFFLVSKLTVDKSRWQQGNVFSCSVMHEALHNHYTQKSLS | |
| | LSP | |
| 22 | QVQLVQSGAEVKKPGASVKVSCKASGYSFTGYYMHWVRQAPGQGLEWIGRINPYN | First polypeptide |
| | GATTYSQNFKDRATMTVDTSISTAYMELSRLRSDDTAVYYCVPTGMGYFLMDYWG | chain |
| | QGTLVTVSSASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKKVEPKSCDKTH | |
| | TSPPSPEVQLLESGGGLVQPGGSLKLSCAASGFTFNTYAMNWVRQAPGKGLEWVAR | |
| | IRSKYNNYATYYADSVKDRFTISRDDSKNTAYLQMNNLKTEDTAVYYCVRHGNFGN | |
| | SYVSWFAYWGQGTLVTVSSGGGGSGGGGSGGGGSELVVTQEPSLTVSPGGTVTLTC | |
| | RSSTGAVTTSNYANWVQQKPGQAPRGLIGGTNKRAPGTPARFSGSLLGGKAALTLSG | |
| | VQPEDEAEYYCALWYSNLWVFGGGTKLTVLGGGGSPKSSDKTHTCPPCPAPEAAGG | |
| | PSVFLFPPKPKDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKAKGQPREPQVY | |
| | TLPPCREEMTKNQVSLWCLVKGFYPSDIAVEWESNGQPENNYKTTPPVLDSDGSFFL | |
| | YSKLTVDKSRWQQGNVFSCSVMHEALHNHYTQKSLSLSP | |
| 23 | QVQLVQSGAEVKKPGASVKVSCKASGYSFTGYYMHWVRQAPGQGLEWIGRINPYN | Second |
| | GATTYSQNFKDRATMTVDTSISTAYMELSRLRSDDTAVYYCVPTGMGYFLMDYWG | polypeptide |
| | QGTLVTVSSASTKGPSVFPLAPSSKSTSGGTAALGCLVKDYFPEPVTVSWNSGALTSG | chain |
| | VHTFPAVLQSSGLYSLSSVVTVPSSSLGTQTYICNVNHKPSNTKVDKKVEPKSCDKTH | |
| | TCPPCPAPEAAGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALPAPIEKTIS | |
| | KAKGQPREPQVCTLPPSREEMTKNQVSLSCAVKGFYPSDIAVEWESNGQPENNYKTT | |
| | PPVLDSDGSFFLVSKLTVDKSRWQQGNVFSCSVMHEALHNHYTQKSLSLSP | |
| 24 | DIQLTQSPSFLSASVGDRVTITCKASQDVGSAVVWYQQKPGKAPKLLIYWASTRHTG | Third polypeptide |
| | VPSRFSGSGSGTEFTLTISSLQPEDFATYFCQQFTSYPTFGQGTKLEIKRTVAAPSVFIFP | chain, |
| | PSDEQLKSGTASVVCLLNNFYPREAKVQWKVDNALQSGNSQESVTEQDSKDSTYSL | fourth polypeptide |
| | SSTLTLSKADYEKHKVYACEVTHQGLSSPVTKSFNRGEC | chain, or second |
| | | peptide fragment |
| 25 | ASTKGPSVFPLAPSSKSTSGGTAALGCLVKDYFPEPVTVSWNSGALTSGVHTFPAVLQ | CH1 fragment |
| | SSGLYSLSSVVTVPSSSLGTQTYICNVNHKPSNTKVDKKVEPKSCDKTHTSPPSP | |
| 26 | RTVAAPSVFIFPPSDEQLKSGTASVVCLLNNFYPREAKVQWKVDNALQSGNSQESVT | CL fragment |
| | EQDSKDSTYSLSSTLTLSKADYEKHKVYACEVTHQGLSSPVTKSFNRGEC | |
| 27 | QVQLVQSGAEVKKPGASVKVSCKASGYSFTGYYMHWVRQAPGQGLEWIGRINPYN | First peptide |
| | GATTYSQNFKDRATMTVDTSISTAYMELSRLRSDDTAVYYCVPTGMGYFLMDYWG | fragment |
| | QGTLVTVSSASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKKVEPKSCDKTH | |
| | TSPPSP | |
| 28 | QVQLVQSGAEVKKPGASVKVSCKASGYSFTGYYMHWVRQAPGQGLEWIGRINPYN | PDL1 |
| | GATTYSQNFKDRATMTVDTSISTAYMELSRLRSDDTAVYYCVPTGMGYFLMDYWG | (VH + CH1) |
| | QGTLVTVSSASTKGPSVFPLAPSSKSTSGGTAALGCLVKDYFPEPVTVSWNSGALTSG | +CD3 scFv |
| | VHTFPAVLQSSGLYSLSSVVTVPSSSLGTQTYICNVNHKPSNTKVDKKVEPKSCDKTH | |
| | TSPPSPEVQLLESGGGLVQPGGSLKLSCAASGFTFNTYAMNWVRQAPGKGLEWVAR | |
| | IRSKYNNYATYYADSVKDRFTISRDDSKNTAYLQMNNLKTEDTAVYYCVRHGNFGN | |
| | SYVSWFAYWGQGTLVTVSSGGGGSGGGGSGGGGSELVVTQEPSLTVSPGGTVTLTC | |
| | RSSTGAVTTSNYANWVQQKPGQAPRGLIGGTNKRAPGTPARFSGSLLGGKAALTLSG | |
| | VQPEDEAEYYCALWYSNLWVFGGGTKLTVL | |
| 29 | EVQLLESGGGLVQPGGSLKLSCAASGFTFNTYAMNWVRQAPGKGLEWVARIRSKY | CD3 ScFv |
| | NNYATYYADSVKDRFTISRDDSKNTAYLQMNNLKTEDTAVYYCVRHGNFGNSYVS | fragment +Fc |
| | WFAYWGQGTLVTVSSGGGGSGGGGSGGGGSELVVTQEPSLTVSPGGTVTLTCRSST | peptide |
| | GAVTTSNYANWVQQKPGQAPRGLIGGTNKRAPGTPARFSGSLLGGKAALTLSGVQP | fragment |
| | EDEAEYYCALWYSNLWVFGGGTKLTVLGGGGSPKSSDKTHTCPPCPAPEAAGGPSV | |
| | FLFPPKPKDTLMISRTPEVTCVVVDVSHEDPEVKFNWYVDGVEVHNAKTKPREEQY | |
| | NSTYRVVSVLTVLHQDWLNGKEYKCKVSNKALPAPIEKTISKAKGQPREPQVYTLPP | |
| | CREEMTKNQVSLWCLVKGFYPSDIAVEWESNGQPENNYKTTPPVLDSDGSFFLYSKL | |
| | TVDKSRWQQGNVFSCSVMHEALHNHYTQKSLSLSP | |

Hereinafter, the present disclosure will be described in detail with reference to examples, the experimental methods without specifying the specific conditions in the examples or test examples were performed according to conventional conditions.

Hereinafter, the solutions of the present disclosure will be illustrated in conjunction with the following examples. It should be understood by those skilled in the art that the following examples are only used to illustrate the present disclosure and shall not be considered as limiting the scope thereof. Where specific techniques or conditions are not specified in the embodiments, they are performed according to techniques or conditions described in the literature in the art or in accordance with the product specification. The reagents or instruments used without specifying the manufacturer are all conventional products that can be obtained commercially.

### Example 1: Production of Antibody

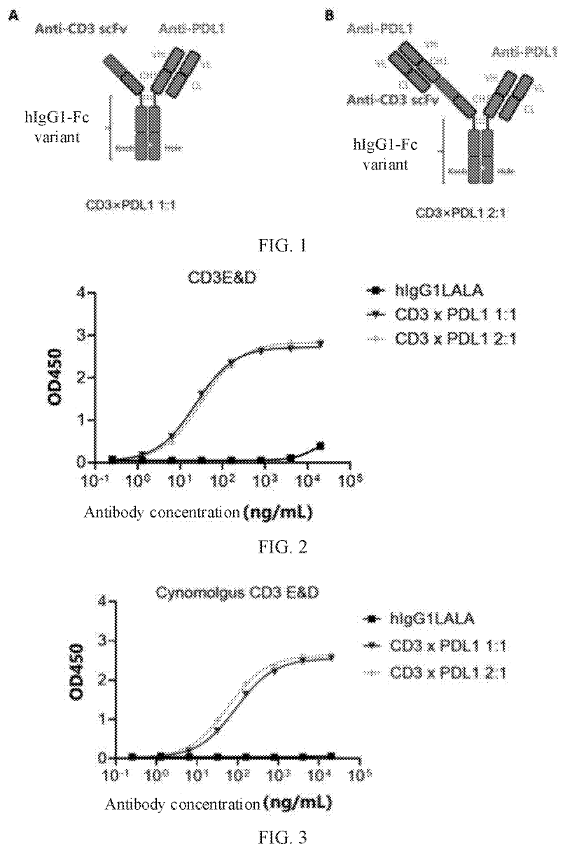

Specific experimental operations for the production of antibody were performed as follows. (1) An ExpiCHO cells (purchased from Thermo Fisher) was cultured using ExpiCHO Expression Medium (purchased from Thermo Fisher), and the cell concentration was adjusted to 6×10⁶/mL to obtain an ExpiCHO cell solution. (2) When the bispecific antibody was in a configuration A (FIG. 1), a pcDNA3.4 vector (synthesized by GenScript Nanjing) including three coding sequences of a CD3 antibody, a PD-L1 antibody heavy chain, and a PD-L1 antibody light chain was added into 2 mL OptiSFM Medium (purchased from Thermo Fisher) at a ratio of 1:1:1 to obtain solution a. Alternatively, when the bispecific antibody was in a configuration B (FIG. 1), the pcDNA3.4 vector (synthesized by GenScript Nanjing) including three coding sequences of PD-L1 heavy chain-CD3 antibody, a PD-L1 antibody heavy chain, and a PD-L1 antibody light chain was added into 2 mL OptiSFM Medium (purchased from Thermo Fisher) at the ratio of 1:1:1 to obtain solution a. (3) A 160 μL of ExpiCHO Expression Medium transfection reagent (purchased from Thermo Fisher) was added into 2 mL of OptiSFM Medium (purchased from Thermo Fisher) to obtain solution b. (4) The solution a and the solution b were then mixed to obtain a transfection mixture, and the entire transfection mixture was added into 50 mL of ExpiCHO cell solution within 5 minutes. (5) After culturing at 37 °C and 5% CO₂ for 1 day, 8 mL of Feed and 300 μL of Enhancer (purchased from Thermo Fisher) were added, and the culture was transferred to 32 °C and 5% CO₂ and cultured for nine days, and then culture supernatant was harvested, wherein 8 mL of Feed was added on day 5. (6) Affinity purification was performed from the culture supernatant using a Protein A purification column (purchased from Nano-Micro). A target antibody was obtained.

Two bispecific antibodies of CD3 and PD-L1 (wherein CD3×PDL1 1:1 is a bispecific antibody with a conventional configuration) were manufactured in this example to investigate various properties of the bispecific antibody of the present disclosure. For the description of the configurations and amino acid sequences of each antibody, reference is made to FIG. 1, Table 1, and Table 2.

TABLE 2
Structures and Corresponding Amino Acid Sequences
of Bispecific Antibody in Example 1

| | Antibody | First Antigen | Second Antigen |
|---|---|---|---|
| Antibody Name | Configuration | Binding Region | Binding Region |
| CD3 × PDL1 1:1 | A | SEQ ID NO: 29 | SEQ ID NO: 23 |
| | | | SEQ ID NO: 24 |
| CD3 × PDL1 2:1 | B | SEQ ID NO: 22 | SEQ ID NO: 23 |
| | | SEQ ID NO: 24 | SEQ ID NO: 24 |

Note:
for the configurations of antibodies of type A and type B, reference is made to FIG. 1.

### Example 2: ELISA Binding Assay of Antibodies of the Present Disclosure

An ELISA assay was used to detect the binding property of the bispecific antibody. An antigen protein was coated onto a 96-well plate. Intensity of a signal after the addition of antibody was used to determine binding property of the bispecific antibody to the antigen protein.

(1) A human PD-L1-His protein, a monkey PD-L1 protein, a human CD3E&D protein, and a monkey CD3E&D protein (purchased from Kactus) were diluted to 2 μg/mL with a PBS buffer, and added to the 96-well plate at a volume of 100 μL/well, followed by incubation overnight at 4 °C. The PBS buffer in the 96-well plate was aspirated, and the plate was washed 6 times with a PBST (pH 7.2 PBS containing 0.1% Tween 20) buffer. Then, 200 μL/well of PBS/10% BSA was added and incubated at 37 °C for 2 h for blocking. The blocking solution was removed, and the plate was washed 6 times with PBST. Then, to-be-tested bispecific antibodies CD3×PD-L1 1:1 (an amino acid sequence of a CD3 single-chain antibody as set forth in SEQ ID NO: 29, an amino acid sequence of a PD-L1 antibody heavy chain as set forth in SEQ ID NO: 23, and an amino acid sequence of the PD-L1 antibody light chain as set forth in SEQ ID NO: 24), CD3×PDL1 2:1 (an amino acid sequence of a PD-L1 antibody heavy chain-CD3 single-chain antibody as set forth in SEQ ID NO: 22, an amino acid sequence of the PD-L1 antibody heavy chain as set forth in SEQ ID NO: 23, and an amino acid sequence of a PD-L1 antibody light chain as set forth in SEQ ID NO: 24) serially diluted with PBST/0.05% BSA, and a control hIgG1LALA (purchased from Biointron) were added at 100 μL/well, followed by incubation at 37 °C for 1 h. A reaction system was removed, and the plate was washed 6 times with PBST. Then, HRP (horseradish peroxidase)-labeled anti-human IgG secondary antibody (purchased from Jackson Lab) diluted with PBST/0.05% BSA was added at 100 μL/well, followed by incubation at 37 °C for 1 h. After washing the plate 6 times with PBST, TMB (tetramethylbenzidine) was added at 80 μL/well, followed by incubation at a room temperature for 3 min. 4 M sulfuric acid was added at 80 μL/well to terminate the reaction. An absorbance value was read at 450 nm using a microplate reader.

The results, as shown in FIG. 2, indicate that both the CD3×PDL1 1:1 antibody and the CD3×PDL1 2:1 antibody were able to bind to the human CD3E&D protein with a comparable capacity.

The results, as shown in FIG. 3, indicate that both the CD3×PDL1 1:1 antibody and the CD3×PDL1 2:1 antibody were able to bind to the monkey CD3E&D protein with the comparable capacity.

The results, as shown in FIG. 4, indicate that both the CD3×PDL1 1:1 antibody and the CD3×PDL1 2:1 antibody were able to bind to the human PDL1-His protein with the comparable capacity.

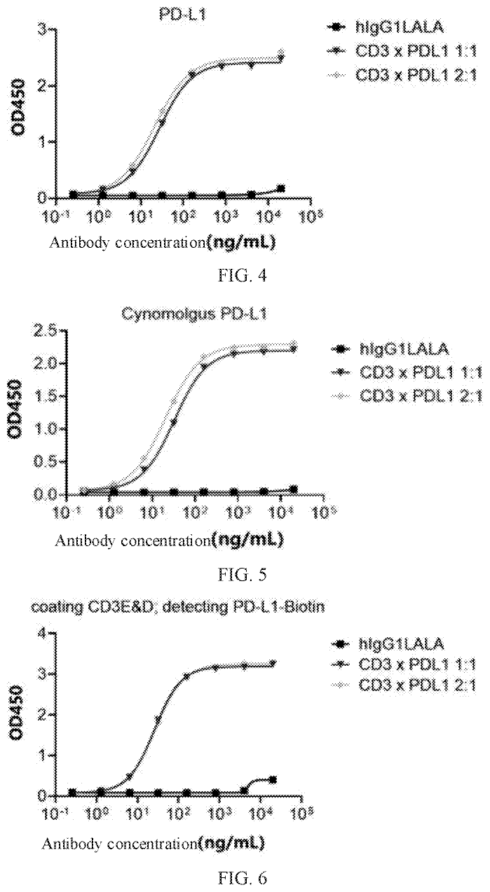

The results, as shown in FIG. 5, indicate that both the CD3×PDL1 1:1 antibody and the CD3×PDL1 2:1 antibody were able to bind to the monkey PDL1 protein with the comparable capacity.

### Example 3: Antibody ELISA Bridging Assay

The ELISA assay was used to detect bridging binding property of the bispecific antibody. A CD3E&D antigen protein was coated onto the 96-well plate. After the addition of antibody, a biotin-labeled PDL1 protein was used for detection. The intensity of the signal was used to determine bridging binding property of the bispecific antibody to CD3E&D and PDL1 proteins.

The CD3E&D protein (purchased from Acro) was diluted to 2 μg/mL with the PBS buffer, and added to the 96-well plate at a volume of 100 μL/well, followed by incubation overnight at 4 °C. The PBS buffer in the 96-well plate was aspirated, and the plate was washed 6 times with PBST (pH 7.2 PBS containing 0.1% Tween 20) buffer. Then, 200 μL/well of PBS/10% BSA was added and incubated at 37 °C for 2 h for blocking. The blocking solution was removed, and the plate was washed 6 times with PBST. Then, to-be-tested bispecific antibodies CD3×PDL1 1:1 (the amino acid sequence of the CD3 single-chain antibody as set forth in SEQ ID NO: 29, the amino acid sequence of the PD-L1 antibody heavy chain as set forth in SEQ ID NO: 23, and the amino acid sequence of the PD-L1 antibody light chain as set forth in SEQ ID NO: 24), CD3×PDL1 2:1 (the amino acid sequence of the PD-L1 antibody heavy chain-CD3 single-chain antibody as set forth in SEQ ID NO: 22, the amino acid sequence of the PD-L1 antibody heavy chain as set forth in SEQ ID NO: 23, and the amino acid sequence of the PD-L1 antibody light chain as set forth in SEQ ID NO: 24) serially diluted with PBST/0.05% BSA, and the control hlgGILALA (purchased from Biointron) were added at 100 μL/well, followed by incubation at 37 °C for 1 h. The reaction system was removed, and the plate was washed 6 times with PBST. Then, PD-L1-Biotin diluted to an appropriate concentration was added, followed by incubation at 37 °C for 1 h. The reaction system was removed, and the plate was washed 6 times with PBST. Then, a HRP (horseradish peroxidase)-labeled streptavidin secondary antibody (purchased from Southern Biotech) diluted with PBST/0.05% BSA was added at 100 μL/well, followed by incubation at 37 °C for 1 h. After washing the plate 6 times with PBST, TMB (tetramethylbenzidine) was added at 80 μL/well, followed by incubation at the room temperature for 3 min. 4 M sulfuric acid was added at 80 μL/well to terminate the reaction. The absorbance value was read at 450 nm using the microplate reader.

The results, as shown in FIG. 6, indicate that both the CD3×PDL1 1:1 antibody and the CD3×PDL1 2:1 antibody were able to bridge CD3E&D and PD-L1 proteins. The CD3×PDL1 1:1 antibody and the CD3×PDL1 2:1 antibody exhibit comparable bridging activities.

### Example 4: Bispecific Antibody Flow Cytometry Binding Assay

A flow cytometry assay was used to detect the binding property of the bispecific antibodies. The antibody was added to a cell. The intensity of the signal after the addition of antibody was used to determine the binding property of the antibody to the cell.
(1) An A-375 human melanoma cell and an A549 human lung cancer cell were diluted to 2×10⁶/mL with PBS and added to a 1.5 mL EP tube at a volume of 100 μL/tube. goat serum was added at 10 μL/tube, followed by blocking at 4 °C for 30 min. Serially diluted bispecific antibodies CD3×PDL1 1:1 (the amino acid sequence of the CD3 single-chain antibody as set forth in SEQ ID NO: 29, the amino acid sequence of the PD-L1 antibody heavy chain as set forth in SEQ ID NO: 23, and the amino acid sequence of the PD-L1 antibody light chain as set forth in SEQ ID NO: 24), CD3×PDL1 2:1 (the amino acid sequence of the PD-L1 antibody heavy chain-CD3 single-chain antibody as set forth in SEQ ID NO: 22, the amino acid sequence of the PD-L1 antibody heavy chain as set forth in SEQ ID NO: 23, and the amino acid sequence of the PD-L1 antibody light chain as set forth in SEQ ID NO: 24), and the control hIgG1LALA (purchased from Biointron) were added, followed by incubation at 4 °C for 30 min. 1 mL of PBS was added to each EP tube, followed by centrifugation at 3500 rpm for 5 min at 4°. The supernatant was completely discarded, and the cells were washed again with PBS. After centrifugation, the supernatant was completely discarded, and the cells were resuspended with PBS at 100 μL/tube. 1 μL/tube of an Alexa-647-labeled goat anti-human IgG secondary antibody (purchased from Jackson Lab) was added, followed by incubation at 4 °C in the dark for 30 min. The cells were washed twice with PBS, and the supernatant was discarded after centrifugation. The cells were resuspended with 200 μL/tube of PBS and analyzed was performed by the flow cytometer.

The results, as shown in FIG. 7, indicate that both the CD3×PDL1 1:1 antibody and the CD3×PDL1 2:1 antibody were able to bind to the A-375 human melanoma cells. In addition, the CD3×PDL1 1:1 antibody and the CD3×PDL1 2:1 antibody exhibit the comparable binding activities.

The results, as shown in FIG. 8, indicate that both the CD3×PDL1 1:1 antibody and the CD3×PDL1 2:1 antibody were able to bind to the A549 human lung cancer cell. In addition, the CD3×PDL1 1:1 antibody and the CD3×PDL1 2:1 antibody exhibit the comparable binding activities.

(2) PBMC was diluted to 2×10⁶/mL with PBS and added to the 1.5 mL EP tube at the volume of 100 μL/tube. 10 μL/tube of goat serum was added, followed by blocking at 4 °C for 30 min. Serially diluted bispecific antibodies CD3×PDL1 1:1 (the amino acid sequence of the CD3 single-chain antibody as set forth in SEQ ID NO: 29, the amino acid sequence of the PDL1 antibody heavy chain as set forth in SEQ ID NO: 23, and the amino acid sequence of the PDL1 antibody light chain as set forth in SEQ ID NO: 24), CD3×PD-L1 2:1 (the amino acid sequence of the PDL1 antibody heavy chain-CD3 single-chain antibody as set forth in SEQ ID NO: 22, the amino acid sequence of the PDL1 antibody heavy chain as set forth in SEQ ID NO: 23, and the amino acid sequence of the PDL1 antibody light chain as set forth in SEQ ID NO: 24), and the control hIgG1LALA (purchased from Biointron) were added, followed by incubation at 4 °C for 30 min. 1 mL of PBS was added to each EP tube, followed by centrifugation at 3500 rpm for 5 min at 4 °C. The supernatant was completely discarded, and the cells were washed again with PBS. After centrifugation, the supernatant was completely discarded, and the cells were resuspended with PBS at 100 μL/tube. 1 μL/tube of Alexa—647-labeled goat anti-human IgG secondary antibody (purchased from Jackson Lab) and 0.5 L/tube of a PerCP-Cy5.5-labeled anti-human CD8 antibody were added, followed by incubation at 4 °C in the dark for 30 min. The cells were washed twice with PBS, and the supernatant was discarded after centrifugation. The cells were resuspended with 200 μL/tube of PBS and analyzed was performed by the flow cytometer.

The results, as shown in FIG. 9, indicate that both the CD3×PDL1 1:1 antibody and the CD3×PDL1 2:1 antibody were able to bind to the T cell. Binding of the CD3×PDL1 2:1 antibody to the T cell was weaker than that of the CD3×PDL1 1:1 antibody of a conventional configuration.

### Example 5: Assay for Bispecific Antibody Promoting PBMC to Kill Tumor Cells

A capacity of the bispecific antibody to promote PBMC to kill the tumor cell was detected.
(1) A complete RPMI-1640 medium was added into a 16-well RTCA plate at a volume of 50 μL per well, and the instrument was calibrated after loading.
(2) A PANC-1 human pancreatic cancer cell, the A-375 human melanoma cell, a HPAF II human pancreatic cancer cell, the A549 human lung cancer cell, a PC-3 human prostate cancer cell, and an HCT-15 human colorectal cancer cell were diluted to 2×10⁵/mL with the complete RPMI-1640 medium, and added separately into the RTCA plate obtained in step (1) at a volume of 50 μL per well respectively. A cell index was then detected for 24 hours by an xCELLigence RTCA MP device under the conditions of 37 °C and 5% CO₂.
(3) The bispecific antibodies CD3×PD-L1 1:1 (the amino acid sequence of the CD3 single-chain antibody as set forth in SEQ ID NO: 29, the amino acid sequence of the PD-L1 antibody heavy chain as set forth in SEQ ID NO: 23, and the amino acid sequence of the PD-L1 antibody light chain as set forth in SEQ ID NO: 24), CD3×PD-L1 2:1 (the amino acid sequence of the PD-L1 antibody heavy chain-CD3 single-chain antibody as set forth in SEQ ID NO: 22, the amino acid sequence of the PD-L1 antibody heavy chain as set forth in SEQ ID NO: 23, and the amino acid sequence of the PD-L1 antibody light chain as set forth in SEQ ID NO: 24) serially diluted with the complete RPMI-1640 medium and the control hlgGILALA (purchased from Biointron) were added into the RTCA plates obtained in step (2) at a volume of 20 μL per well.
(4) PBMC (purchased from Saili Biological) was diluted to 1.25×10⁶/mL with the complete RPMI-1640 medium and added into the RTCA plate obtained in step (3) at a volume of 80 μL per well.
(5) A reaction system obtained in step (4) was placed under the conditions of 37 °C and 5% CO₂, and the cell index was monitored for 24 h by using the xCELLigence RTCA MP device.

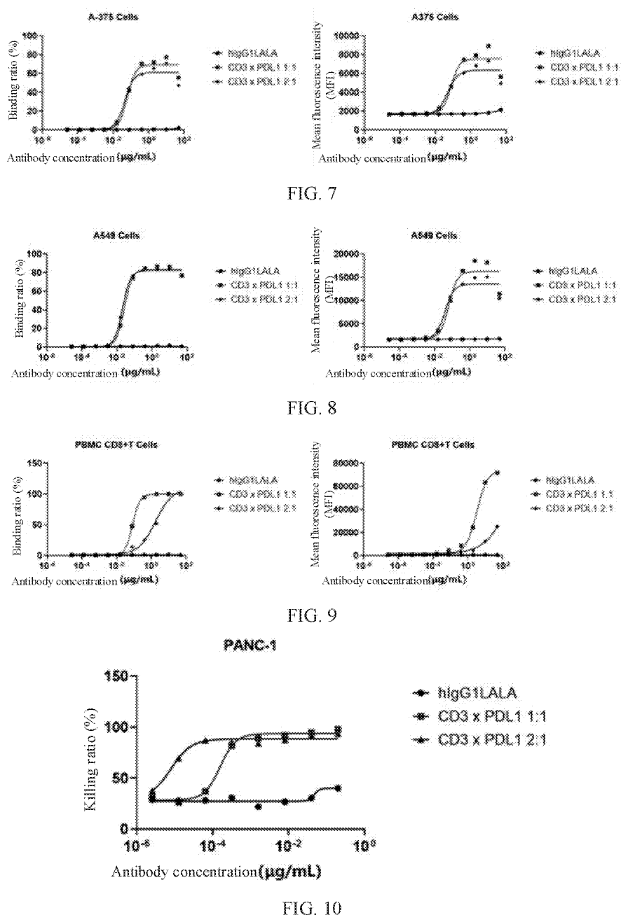

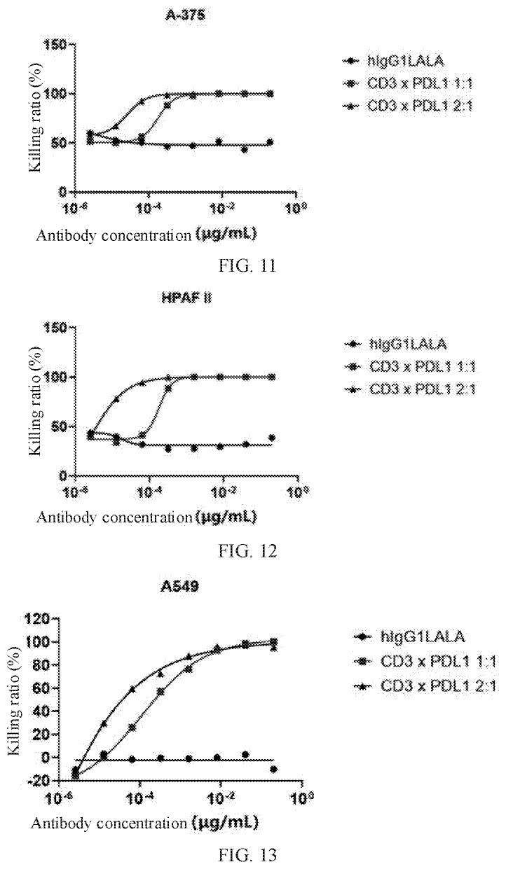

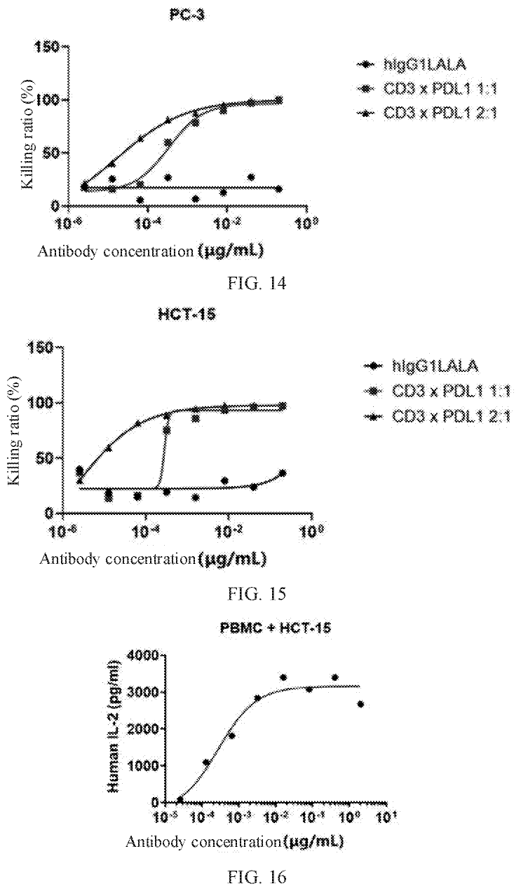

The results, as shown in FIG. 10, FIG. 11, FIG. 12, FIG. 13, FIG. 14, and FIG. 15, indicate that both CD3×PDL1 1:1 and CD3×PDL1 2:1 were capable of promoting PBMC to kill the PANC-1 human pancreatic cancer cell, the A-375 human melanoma cell, the HPAF II human pancreatic cancer cell, the A549 human lung cancer cell, the PC-3 human prostate cancer cell, and the HCT-15 human colorectal cancer cell. It was unexpectedly found that although the capacity of the CD3×PDL1 2:1 antibody binding to the T cell was weakened, a capacity of the CD3×PDL1 2:1 antibody in promoting the T cell to kill the tumor cell was stronger than that of the CD3×PDL1 1:1 antibody of the conventional configuration.

### Example 6: Assay for Bispecific Antibody Promoting PBMC to Secrete Cytokines

The bispecific antibody was added into a co-incubation system of PBMC and the HCT-15 human colorectal cancer cell. A culture supernatant was collected after 48 hours of cultivation. A cytokine content in the supernatant was detected to determine property of the bispecific antibody in inducing cytokine release.
(1) An HCT-15 cell was diluted to 1×10⁵/mL with the complete RPMI 1640 medium, added into the 96-well plate, and cultured in an incubator at 37 °C with 5% CO₂ for 24 hours.
(2) The bispecific antibody CD3×PDL1 2:1 (the amino acid sequence of the PDL1 antibody heavy chain-CD3 single-chain antibody as set forth in SEQ ID NO: 22, the amino acid sequence of the PDL1 antibody heavy chain as set forth in SEQ ID NO: 23, and the amino acid sequence of the PDL1 antibody light chain as set forth in SEQ ID NO: 24) was serially diluted with the complete RPMI 1640 medium and added into the 96-well plate at 20 μL per well.
(3) PBMC (purchased from Saili Biological) was diluted to 1.25×10⁶/mL with the complete RPMI 1640 medium and added into the 96-well plate at 80 μL per well.
(4) The 96-well plate was cultivated in the incubator at 37 °C with 5% CO₂ for 48 hours.
(5) the cells were centrifuged at 300 g for 10 minutes at the room temperature, and cell culture supernatant was collected.
(6) The cytokine content in the supernatant was detected by using a CBA kit (purchased from BD).

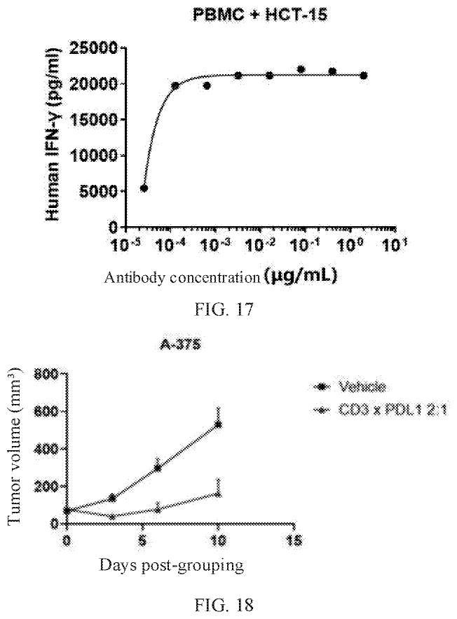

The results, as shown in FIG. 16 and FIG. 17, indicating that CD3×PDL1 2:1 was able to activate PBMC and induce secretion of cytokines IL-2 and IFN-γ in the presence of the target cell.

The above results indicate that the bispecific antibody of the 2:1 configuration (Configuration B) in the present disclosure has the capacity to promote PBMC to secrete an immune activating cytokine.

### Example 7: Anticancer Assay of Bispecific Antibody in Mouse Model

In vivo pharmacodynamic experiment was used to detect the anti-cancer function of the bispecific antibody of the present disclosure in promoting immune reconstitution of mice.
(1) On day-8, each NSG mouse was injected with 5×10⁶ A-375 human melanoma cells with the tumor bearing on the right ventral side of the NSG mice.
(2) On day-8, the human PBMC (purchased from Saili Biological) was transfused into the NSG mouse (purchased from Southern Model Animal Center) via a tail vein at a dosage of 5×10⁶ cells per mouse.
(3) On day 0, the mouse was weighed and a tumor volume was measured. The mice were divided into control groups and treatment groups according to obtained body weight results and tumor volume results.
(4) On day 0, day 3, and day 6, the CD3×PD-L1 2:1 (the amino acid sequence of the PDL1 antibody heavy chain-CD3 single-chain antibody as set forth in SEQ ID NO: 22, the amino acid sequence of the PD-L1 antibody heavy chain as set forth in SEQ ID NO: 23, and the amino acid sequence of the PD-L1 antibody light chain as set forth in SEQ ID NO: 24) and solvent control PBS were injected into the mice via the tail vein at a volume of 250 μL per mouse.
(5) The tumor volume was measured, and the mice were weighed twice every week after antibody injection.

The results, as shown in FIG. 18, indicate that CD3×PDL1 2:1 administered at a dose of 0.1 mg/kg exhibits significant anticancer activity.

The above assay results show that the bispecific antibody of the present disclosure can bind to CD3 and PDL1, thereby promoting T cell activation and cytokine secretion, effectively promoting PBMC to kill the tumor cell, and exhibiting good anticancer activity. The bispecific antibody of the present disclosure can achieve high-affinity binding to the tumor cell and low-affinity binding to the T cell and has higher anticancer activity and higher safety. In summary, the bispecific antibody of the present disclosure can promote the immune cell to fight the cancer, have good anticancer activity and higher safety, and have high values in clinical application and drug development

The preferred embodiments of the present disclosure are described in detail above, but the present disclosure is not limited thereto. Within the scope of the technical concept of the present disclosure, various simple modifications can be made to the technical solution of the present disclosure, including the combination of various technical features in any other suitable manner. These simple modifications and combinations shall also be regarded as the content disclosed by the present disclosure, and all fall within the protection scope of the present disclosure.

In the description of the present disclosure, reference to the terms “one embodiment,” “some embodiments,” “an example,” “a specific example,” or “some examples,” etc., means that the specific features, structures, materials, or characteristics described in conjunction with the embodiment(s) or example(s) are included in at least one embodiment or example of the present disclosure. In the present disclosure, any illustrative description of the above terms does not necessarily refer to the same embodiment(s) or example(s). Further, the particular features, structures, materials, or characteristics may be combined in any one or more embodiments or examples in any suitable manner. In addition, different embodiments or examples and features of different embodiments or examples described in the specification may be combined by those skilled in the art without mutual contradiction.

Although the embodiments of the present disclosure are illustrated and described above, it should be understood that the above-mentioned embodiments are exemplary and should not be construed as limiting the present disclosure. Those skilled in the art can make changes, modifications, substitutions, and variations to the above-mentioned embodiments within the scope of the present disclosure.

What is claimed is:

1. A bispecific antibody, comprising:
a first antigen binding region;
a second antigen binding region; and
an Fc portion, wherein:
the Fc portion comprises a first Fc peptide fragment and a second Fc peptide fragment;
the first antigen binding region comprises a scFv fragment, and a binding protein of a first molecule or fragment thereof, wherein the binding protein of the first molecule or fragment thereof is linked to an N-terminus of the scFv fragment, and wherein a C-terminus of the scFv fragment is linked to an N-terminus of the first Fc peptide fragment;
the second antigen binding region comprises a binding protein of a second molecule or fragment thereof, wherein the binding protein of the second molecule or fragment thereof is linked to an N-terminus of the second Fc peptide fragment; and
the first molecule and the second molecule are PD-L1, and the scFv fragment has CD3 binding activity.

2. The bispecific antibody according to claim 1, wherein the binding protein of the first molecule or fragment thereof and/or the binding protein of the second molecule or fragment thereof are each independently selected from at least one of a Fab fragment or an F(ab′)₂ fragment.

3. The bispecific antibody according to claim 1, wherein:
the binding protein of the first molecule or fragment thereof is a first Fab fragment; and/or
the binding protein of the second molecule or fragment thereof is a second Fab fragment.

4. The bispecific antibody according to claim 3, wherein each of the first Fab fragment and the second Fab fragment comprises:
a first heavy chain variable region CDR1, the amino acid sequence of the first heavy chain variable region CDR1 being set forth in SEQ ID NO: 1;
a first heavy chain variable region CDR2, the amino acid sequence of the first heavy chain variable region CDR2 being set forth in SEQ ID NO: 2;
a first heavy chain variable region CDR3, the amino acid sequence of the first heavy chain variable region CDR3 being set forth in SEQ ID NO: 3;
a first light chain variable region CDR1, the amino acid sequence of the first light chain variable region CDR1 being set forth in SEQ ID NO: 4;
a first light chain variable region CDR2, the amino acid sequence of the first light chain variable region CDR2 being set forth in SEQ ID NO: 5; and
a first light chain variable region CDR3, the amino acid sequence of the first light chain variable region CDR3 being set forth in SEQ ID NO: 6.

5. The bispecific antibody according to claim 3, wherein the first Fab fragment and/or the second Fab fragment comprises a first heavy chain framework region and/or a first light chain framework region; and
wherein each of the first Fab fragment and the second Fab fragment comprises:
a first peptide fragment having an amino acid sequence as set forth in SEQ ID NO: 27; and
a second peptide fragment having an amino acid sequence as set forth in SEQ ID NO: 28.

6. The bispecific antibody according to claim 4, wherein:
the first heavy chain variable regions of the first Fab fragment and the second Fab fragment are each independently selected from an amino acid sequence as set forth in SEQ ID NO: 7 or an amino acid sequence having at least 90% identity thereto; and/or
the first light chain variable regions of the first Fab fragment and the second Fab fragment are each independently selected from an amino acid sequence as set forth in SEQ ID NO: 8 or an amino acid sequence having at least 90% identity thereto.

7. The bispecific antibody according to claim 4, wherein the first Fab fragment and/or the second Fab fragment further comprises:
a CH1 fragment, wherein a C-terminus of the first heavy chain variable region is linked to an N-terminus of the CH1 fragment; and
a CL fragment, wherein a C-terminus of the first light chain variable region is linked to an N-terminus of the CL fragment,
wherein the CH1 fragment is linked to the CL fragment via an interchain disulfide bond.

8. The bispecific antibody according to claim 7, wherein:
the CH1 fragment has an amino acid sequence as set forth in SEQ ID NO: 25;
the CL fragment has an amino acid sequence as set forth in SEQ ID NO: 26; and
a C-terminus of the CH1 fragment of the first Fab fragment is linked to the N-terminus of the scFv fragment, and the C-terminus of the scFv fragment is linked to the N-terminus of the first Fc peptide fragment; and/or a C-terminus of the CH1 fragment of the second Fab fragment is linked to the N-terminus of the second Fc peptide fragment.

9. The bispecific antibody according to claim 1, wherein the scFv fragment comprises:
a second heavy chain variable region CDR1, the amino acid sequence of the second heavy chain variable region CDR1 being set forth in SEQ ID NO: 9;
a second heavy chain variable region CDR2, the amino acid sequence of the second heavy chain variable region CDR2 being set forth in SEQ ID NO: 10;
a second heavy chain variable region CDR3, the amino acid sequence of the second heavy chain variable region CDR3 being set forth in SEQ ID NO: 11;
a second light chain variable region CDR1, the amino acid sequence of the second light chain variable region CDR1 being set forth in SEQ ID NO: 12;
a second light chain variable region CDR2, the amino acid sequence of the second light chain variable region CDR2 being set forth in SEQ ID NO: 13; and
a second light chain variable region CDR3, the amino acid sequence of the second light chain variable region CDR3 being set forth in SEQ ID NO: 14.

10. The bispecific antibody according to claim 1, wherein the scFv fragment comprises a second heavy chain framework region and/or a second light chain framework region; and
wherein the scFv fragment comprises a second heavy chain variable region having an amino acid sequence as set forth in SEQ ID NO: 15 or an amino acid sequence having at least 90% identity thereto; and a second light chain variable region having an amino acid sequence as set forth in SEQ ID NO: 16 or an amino acid sequence having at least 90% identity thereto.

11. The bispecific antibody according to claim 10, wherein the scFv fragment further comprises a linker peptide 1, wherein:
an N-terminus of the linker peptide 1 is linked to a C-terminus of the second heavy chain variable region, and a C-terminus of the linker peptide 1 is linked to an N-terminus of the second light chain variable region; or
the N-terminus of the linker peptide 1 is linked to a C-terminus of the second light chain variable region, and the C-terminus of the linker peptide 1 is linked to an N-terminus of the second heavy chain variable region; and
wherein the linker peptide 1 has an amino acid sequence as set forth in (GGGGS) n, where n is an integer greater than or equal to 1.

12. The bispecific antibody according to claim 11, wherein the scFv fragment has an amino acid sequence as set forth in SEQ ID NO: 17.

13. The bispecific antibody according to claim 11, wherein the linker peptide 1 has an amino acid sequence as set forth in SEQ ID NO: 18.

14. The bispecific antibody according to claim 11, wherein the first antigen binding region further comprises a linker peptide 2, wherein a C-terminus of the CH1 fragment of the first Fab fragment is linked to an N-terminus of the linker peptide 2, and wherein a C-terminus of the linker peptide 2 is linked to the N-terminus of the scFv fragment; and
wherein the linker peptide 2 has an amino acid sequence as set forth in (GGGGS) n, where n is an integer greater than or equal to 1.

15. The bispecific antibody according to claim 14, wherein the linker peptide 2 has an amino acid sequence as set forth in SEQ ID NO: 19.

16. The bispecific antibody according to claim 1, wherein each of the first Fc peptide fragment and the second Fc peptide fragment is a human Fc peptide fragment; or
the first Fc peptide fragment has an amino acid sequence as set forth in SEQ ID NO: 20, and wherein the second Fc peptide fragment has an amino acid sequence as set forth in SEQ ID NO: 21.

17. The bispecific antibody according to claim 2, comprising:
a first polypeptide chain having an amino acid sequence as set forth in SEQ ID NO: 22 or an amino acid sequence having at least 80% identity thereto;
a second polypeptide chain having an amino acid sequence as set forth in SEQ ID NO: 23 or an amino acid sequence having at least 80% identity thereto;
a third polypeptide chain having an amino acid sequence as set forth in SEQ ID NO: 24 or an amino acid sequence having at least 80% identity thereto; and
a fourth polypeptide chain having the amino acid sequence as set forth in SEQ ID NO: 24 or the amino acid sequence having at least 80% identity thereto.

18. The bispecific antibody according to claim 2, comprising:
a first polypeptide chain having an amino acid sequence as set forth in SEQ ID NO: 22;
a second polypeptide chain having an amino acid sequence as set forth in SEQ ID NO: 23;
a third polypeptide chain having an amino acid sequence as set forth in SEQ ID NO: 24; and
a fourth polypeptide chain having an amino acid sequence as set forth in SEQ ID NO: 24.

19. A pharmaceutical composition comprising the bispecific antibody according to claim 1.

20. A method for preventing and/or treating a CD3-mediated disease and/or a PD-L1-mediated disease, comprising administering to a subject a pharmaceutically acceptable dosage of the bispecific antibody according to claim 1.


SEQUENCE LISTING (29 sequences)

SEQ ID NO: 1 - protein, 8 aa, synthetic construct
GYSFTGYY

SEQ ID NO: 2 - protein, 8 aa, synthetic construct
INPYNGAT

SEQ ID NO: 3 - protein, 12 aa, synthetic construct
VPTGMGYFLMDY

SEQ ID NO: 4 - protein, 6 aa, synthetic construct
QDVGSA

SEQ ID NO: 5 - skipped

SEQ ID NO: 6 - protein, 8 aa, synthetic construct
QQFTSYPT

SEQ ID NO: 7 - protein, 119 aa, synthetic construct
QVQLVQSGAEVKKPGASVKVSCKASGYSFTGYYMHWVRQAPGQGLEWIGRINPYNGATTYSQNFKDRATMTVDTSISTAYMELSRLRSDDTAVYYCVPTGMGYFLMDYWGQGTLVTVSS

SEQ ID NO: 8 - protein, 106 aa, synthetic construct
DIQLTQSPSFLSASVGDRVTITCKASQDVGSAVVWYQQKPGKAPKLLIYWASTRHTGVPSRFSGSGSGTEFTLTISSLQPEDFATYFCQQFTSYPTFGQGTKLEIK

SEQ ID NO: 9 - protein, 8 aa, synthetic construct
GFTFNTYA

SEQ ID NO: 10 - protein, 10 aa, synthetic construct
IRSKYNNYAT

SEQ ID NO: 11 - protein, 16 aa, synthetic construct
VRHGNFGNSYVSWFAY

SEQ ID NO: 12 - protein, 9 aa, synthetic construct
TGAVTTSNY

SEQ ID NO: 13 - skipped

SEQ ID NO: 14 - protein, 9 aa, synthetic construct
ALWYSNLWV

SEQ ID NO: 15 - protein, 125 aa, synthetic construct
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 16 - protein, 109 aa, synthetic construct
ELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 17 - protein, 249 aa, synthetic construct
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 18 - protein, 15 aa, synthetic construct
GGGGSGGGGSGGGGS

SEQ ID NO: 19 - protein, 5 aa, synthetic construct
GGGGS

SEQ ID NO: 20 - protein, 229 aa, synthetic construct
PKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 21 - protein, 229 aa, synthetic construct
PKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 22 - protein, 715 aa, synthetic construct
QVQLVQSGAEVKKPGASVKVSCKASGYSFTGYYMHWVRQAPGQGLEWIGRINPYNGATTYSQNFKDRATMTVDTSISTAYMELSRLRSDDTAVYYCVPTGMGYFLMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPEVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 23 - protein, 447 aa, synthetic construct
QVQLVQSGAEVKKPGASVKVSCKASGYSFTGYYMHWVRQAPGQGLEWIGRINPYNGATTYSQNFKDRATMTVDTSISTAYMELSRLRSDDTAVYYCVPTGMGYFLMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 24 - protein, 213 aa, synthetic construct
DIQLTQSPSFLSASVGDRVTITCKASQDVGSAVVWYQQKPGKAPKLLIYWASTRHTGVPSRFSGSGSGTEFTLTISSLQPEDFATYFCQQFTSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 113 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSP

SEQ ID NO: 26 - protein, 107 aa, synthetic construct [sequence omitted]

SEQ ID NO: 27 - protein, 232 aa, synthetic construct
QVQLVQSGAEVKKPGASVKVSCKASGYSFTGYYMHWVRQAPGQGLEWIGRINPYNGATTYSQNFKDRATMTVDTSISTAYMELSRLRSDDTAVYYCVPTGMGYFLMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSP

SEQ ID NO: 28 - protein, 481 aa, synthetic construct
QVQLVQSGAEVKKPGASVKVSCKASGYSFTGYYMHWVRQAPGQGLEWIGRINPYNGATTYSQNFKDRATMTVDTSISTAYMELSRLRSDDTAVYYCVPTGMGYFLMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPEVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 29 - protein, 483 aa, synthetic construct
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP